(19) United States
(12) Patent Application Publication
(10) Pub. No.: US 2015/0265675 A1
(43) Pub. Date: Sep. 24, 2015

(54) Treatment of Brain Injury or Trauma with TSG-6 Protein

(71) Applicants: Darwin J. Prockop, Philadelphia, PA (US); Dong-Ki Kim, Temple, TX (US); Jun Watanabe, Kawasaki (JP); Ashok Shetty, Austin, TX (US); Bharathi Hattiangady, Belton, TX (US); Jessica Foraker, Seattle, WA (US); Barry Berkowitz, Framingham, MA (US)

(72) Inventors: Darwin J. Prockop, Philadelphia, PA (US); Dong-Ki Kim, Temple, TX (US); Jun Watanabe, Kawasaki (JP); Ashok Shetty, Austin, TX (US); Bharathi Hattiangady, Belton, TX (US); Jessica Foraker, Seattle, WA (US); Barry Berkowitz, Framingham, MA (US)

(21) Appl. No.: 14/435,800

(22) PCT Filed: Oct. 17, 2013
(86) PCT No.: PCT/US13/65349

§ 371 Date: Apr. 15, 2015

Related U.S. Application Data
(60) Provisional application No. 61/714,859, filed on Oct. 17, 2012.

(51) Int. Cl. A61K 38/17; A61K 45/06

(52) U.S. Cl. CPC A61K 38/1709; A61K 45/06

(57) ABSTRACT

A method of treating brain injury or brain trauma in an animal by administering to an animal TSG-6 protein or a biologically active fragment, derivative, or analogue thereof.

This application claims priority based on provisional Application Ser. No. 61/714,859, filed Oct. 17, 2012, the contents of which are incorporated by reference in their entirety.

This invention relates to the treatment of brain injury or brain trauma with inflammation modulatory or anti-inflammatory proteins. More particularly, this invention relates to the treatment of brain injury or brain trauma in an animal (including humans) by administering to the animal TSG-6 protein or a biologically active fragment, derivative, or analogue thereof.

Traumatic brain injury (TBI) is a leading cause of death and disability in young adults and children in the developed countries of the world (Coronado et al., 2011), who fall victim to motor vehicle accidents, falls, sports injuries, and physical assaults. The CDC has described TBI as a serious public health problem in the United States. Each year, TBI contributes to a substantial number of deaths and cases of permanent disability. Recent data show that, on average, about 1.7 million people sustain a traumatic brain injury annually. Many individuals who survive TBI experience acute or chronic deficits in motor, cognitive, behavioral, or social function (Thurman et al., 1999). TBI may be of the closed or penetrating head injury types.

Aside from the enormous personal burden, TBI generates substantial economic costs, estimated to be more than $55 billion dollars per year in the United States (Maas et al., 2008). Despite advances in clinical care, a vast majority of the survivors of severe TBI are not able to live independently with a loss of working memory the most troubling symptom cited by patients (Myburgh et al., 2008). TBI invokes a complex inflammatory response by the innate immune system, mediated primarily through microglia and astrocytes but also involving cross-talk with invading neutrophils, macrophages, and T cells (Ransohoff and Brown, 2012). The inflammatory response is both harmful and helpful in that it can cause excessive destruction of tissues, and it clears necrotic and apoptotic cells. A number of anti-inflammatory agents have been tested in TBI but all have been disappointing. Glucorticoids were used clinically to decrease brain edema but failed in a large clinical trial because of increased mortality (Edwards et al., 2005; Roberts et al., 2004). Also, glucocorticoids were shown to aggravate retrograded memory deficits in a TBI model (Chen et al., 2009). Non-steroidal anti-inflammatory drugs produced mixed results in models for TBI with some reports indicating improvements (Kovesdi et al., 2012; Thau Zuchman et al., 2012), and others indicating deleterious effects such as worsened cognitive outcomes (Browne et al., 2006). Strategies to reduce inflammation by targeting toll-like receptor (TLR) ligands, TLR receptors or pro-inflammatory cytokines also have proven ineffective. (Rivest, 2011).

Mesenchymal stem/stromal cells (MSCs) offer potentially a novel therapy for multiple central nervous system pathologies such as stroke, Parkinson's Disease, experimental autoimmune encephalomyelitis, and amyotrophic lateral sclerosis, and TBI (Parr et al., 2007; Prockop, 2007; Chopp et al., 2008; Zietlow et al., 2008, Uccelli and Prockop, 2010). MSCs initially attracted interest for their ability to differentiate into multiple cellular phenotypes in culture and in vivo (Kopen et al., 1999; Parr et al., 2007); however, recent observations indicate that only small numbers of the cells engraft into most injured tissues (Harting et al., 2009), and they disappear quickly (Munoz et al., 2005; Schrepfer et al., 2007). Previous studies have demonstrated that human MSCs enhance repair of the damaged brain in part through modulations of neuro-inflammation (Ohtaki et al., 2008; Foraker et al., 2012). Previous reports showed that intravenous infusion of MSCs to a rodent model of TBI suppressed leakage through the blood brain barrier (BBB) and decreased neural damage (Mahmood et al., 2004; Pati et al., 2011); however, the mechanism and therapeutic factors produced by MSCs were not defined.

We showed that MSCs suppressed endotoxin-induced glial activation in organotypic hippocampal slice cultures (Foraker et al., 2012). More recently, we reported that some of the therapeutic effects of MSCs can be explained by activation of the cells to express the inflammation modulatory protein TSG-6 in animal models for myocardial infarction (Lee et al., 2009), peritonitis (Choi et al., 2011) and chemical injury of the cornea (Oh et al., 2010). TSG-6 is a multifunctional protein that normally is up-regulated in many pathological contexts (Getting et al., 2002; Mahoney et al., 2005; Milner and Day, 2003; Szanto et al., 2004; Wisniewski et al., 1996). The protein has multiple effects on the inflammatory response, including modulation of TLR2/TNF-α signaling in resident macrophages during the initial mild phase of inflammation (Phase I inflammation) (Choi et al., 2011; Oh et al., 2010). As a result, the protein decreased the secondary cytokine storm that is triggered by resident macrophages and that ushers in the massive inflammatory response to tissue injury (Phase II inflammation) (Choi et al., 2011; Oh et al., 2010). In a mouse model for chemical injuries of the cornea (Oh et al., 2010), administration of TSG-6 during the Phase I of inflammation, but not during Phase II, effectively suppressed the Phase II response and prevented pathological changes. Chemical injuries of the cornea resemble TBI in that steroids are used in caution and other anti-inflammatory agents are contraindicated because they activate proteases that cause melting of the corneal stroma (Flach, 2000; Lin et al., 2000). Here we demonstrated that i.v. injection of TSG-6 reduced markedly disruption of the BBB and loss of brain tissue following cortical contusion injury in mice. We found TSG-6 decreased neutrophil infiltration and thereby decreased MMP-9 activity and protected the brain against secondary damage.

In accordance with an aspect of the present invention, there is provided a method of treating a brain injury or brain trauma in an animal. The method comprises administering to the animal at least one inflammation modulatory or anti-inflammatory protein or polypeptide or biologically active fragment, derivative, or analogue thereof. The at least one inflammation modulatory or anti-inflammatory protein or polypeptide is administered in an amount effective to treat the brain injury or brain trauma in the a nimal.

In a non-limiting embodiment, the at least one inflammation modulatory or anti-inflammatory protein or polypeptide is tumor necrosis factor-α stimulating gene 6 (TSG-6) protein or a biologically active fragment, derivative, or analogue thereof.

In a non-limiting embodiment, the TSG-6 protein is the “native” TSG-6 protein, which has 277 amino acid residues as shown hereinbelow.

MIILIYLFLL LWEDTQGWGF KDGIFHNSIW LERAAGVYHR
EARSGKYKLT YAEAKAVCEF EGGHLATYKQ LEAARKIGFH
VCAAGWMAKG RVGYPIVKPG PNCGFGKTGI IDYGIRLNRS
ERWDAYCYNP HAKECGGVFT DPKQIFKSPG FPNEYEDNQI
CYWHIRLKYG QRIHLSFLDF DLEDDPGCLA DYVEIYDSYD
DVHGFVGRYC GDELPDDIIS TGNVMTLKFL SDASVTAGGF
QIKYVAMDPV SKSSQGKNTS TTSTGNKNFL AGRFSHL

In another non-limiting embodiment, the at least one inflammation modulatory or anti-inflammatory protein or polypeptide is a fragment of TSG-6 protein known as a TSG-6-LINK protein, or a TSG-6 link module domain. In one non-limiting embodiment, the TSG-6 link module domain consists of amino acid residues 1 through 133 of the above-mentioned sequence.

In another non-limiting embodiment, the TSG-6 link module domain consists of amino acid residues 1 through 98 of the above-mentioned sequence and is described in Day, et al., Protein Expr. Purif., Vol. 8, No. 1, pgs. 1-16 (August 1996).

In another non-limiting embodiment, the at least one inflammation modulatory or anti-inflammatory protein or polypeptide or a biologically active fragment, derivative, or analogue thereof, such as TSG-6 protein or a biologically active fragment, derivative, or analogue thereof, has a “His-tag” at the C-terminal thereof. The term “His-tag”, as used herein, means that one or more histidine residues are bound to the C-terminal of the TSG-6 protein or biologically active fragment, derivative, or analogue thereof. In another non-limiting embodiment, the “His-tag” has six histidine residues at the C-terminal of the biologically active protein or polypeptide, such as TSG-6 protein or a biologically active fragment, derivative, or analogue thereof.

In a non-limiting embodiment, when the inflammation modulatory or anti-inflammatory protein or polypeptide, or biologically active fragment, derivative, or analogue thereof, includes a “His-tag”, at the C-terminal thereof, the inflammation modulatory or anti-inflammatory protein or polypeptide, or biologically active fragment, derivative, or analogue thereof, may include a cleavage site that provides for cleavage of the “His-tag” from the inflammation modulatory or anti-inflammatory protein or polypeptide, or biologically active fragment, derivative, or analogue thereof, after the inflammation modulatory or anti-inflammatory polypeptide, or biologically active fragment, derivative, or analogue thereof is produced.

In yet another non-limiting embodiment, the at least one inflammation modulatory or anti-inflammatory protein or polypeptide, or a biologically active fragment, derivative, or analogue thereof, such as TSG-6 protein or a biologically active fragment, derivative, or analogue thereof, is bound, conjugated, or otherwise attached to at least one molecule that enhances the biological activity and/or residence time of the at least one inflammation modulatory or anti-inflammatory protein or polypeptide. In a non-limiting embodiment, such at least one molecule is polyethylene glycol, or PEG.

The at least one inflammation modulatory or anti-inflammatory protein or polypeptide may be made by techniques known to those skilled in the art. In a non-limiting embodiment, the at least one inflammation modulatory or anti-inflammatory protein or polypeptide may be prepared recombinantly by genetic engineering techniques known to those skilled in the art. In another non-limiting embodiment, the at least one inflammation modulatory or anti-inflammatory protein or polypeptide may be synthesized on an automatic peptide synthesizer.

In a non-limiting embodiment, the at least one inflammation modulatory or anti-inflammatory protein or polypeptide is administered systemically, such as by intravenous, intraarterial, or intraperitoneal administration, or the at least one inflammation modulatory or anti-inflammatory protein or polypeptide may be administered directly to the site of brain trauma or injury. In a non-limiting embodiment, the at least one inflammation modulatory or anti-inflammatory protein or polypeptide is administered intravenously. In another non-limiting embodiment, the at least one inflammation modulatory or anti-inflammatory protein or polypeptide is administered directly to the site of brain trauma or injury.

In another non-limiting embodiment, the at least one inflammation modulatory or anti-inflammatory protein or polypeptide may be coated onto a stent, which is delivered to a blood vessel that is located at the site of the brain trauma or injury.

The at least one inflammation modulatory or anti-inflammatory protein or polypeptide may be administered to any animal that has suffered brain injury or trauma, including mammals, birds, reptiles, amphibians, and fish. In a non-limiting embodiment, the animal is a mammal. In another non-limiting embodiment, the mammal is a primate, which includes human and non-human primates.

The at least one inflammation modulatory or anti-inflammatory protein or polypeptide may be administered in conjunction with an acceptable pharmaceutical carrier or excipient.

Suitable carriers and excipients include those that are compatible physiologically and biologically with the inflammation modulatory or anti-inflammatory protein or polypeptide and with the patient, such as phosphate buffered saline and other suitable carriers or excipients. Other pharmaceutical carriers that may be employed, either alone or in combination, include, but are not limited to, sterile water, alcohol, fats, waxes, and inert solids. Pharmaceutically acceptable adjuvants (e.g., buffering agent, dispersing agents) also may be incorporated into a pharmaceutical composition including the anti-inflammatory protein or polypeptide. In general, compositions useful for parenteral administration are well known. (See, for example, Remington's Pharmaceutical Science, 17^{th} Ed., Mack Publishing Co., Easton, Pa., 1990).

Brain injuries which may be treated include, but are not limited to, any traumatic brain injury caused by trauma to the brain, including, but not limited to, striking of the head with solid objects, falls, contusions, concussions, including brain injury caused by repeated concussions, such as those that may be suffered by those participating in sports, such as football, baseball, basketball, wrestling, skiing, horse racing, auto racing, and hockey, and brain injuries caused by explosions resulting from explosive devices including, but not limited to, incendiary explosive devices (IEDs). There may or may not be penetration of the head or brain. Such traumatic brain injuries also include, but are not limited to, any brain injury resulting from diseases or disorders of the brain, including, but not limited to, stroke, Parkinson's Disease, autoimmune encephalitis, amyotrophic lateral sclerosis (Lou Gehrig's Disease or ALS), for example. It is to be understood, however, that the scope of the present invention is not to be limited to the treatment of any particular brain injury or trauma.

The at least one inflammation modulatory or anti-inflammatory protein or polypeptide, or biologically active fragment, derivative, or analogue thereof, is administered in an amount effective to treat brain injury or brain trauma in an animal. In a non-limiting embodiment, the at least one inflammation modulatory or anti-inflammatory protein or polypeptide, or biologically active fragment, derivative, or analogue thereof, is administered in a total amount of from about 10 μg to about 100 μg. In another non-limiting embodiment, the at least one inflammation modulatory or anti-inflammatory protein or polypeptide, or biologically active fragment, derivative, or analogue thereof, is administered in a total amount of from about 50 μg to about 100 μg. The exact amount of inflammation modulatory or anti-inflammatory protein or polypeptide or fragment, derivative, or analogue thereof to be administered is dependent on a variety of factors, including, but not limited to, the age, weight, and sex of the patient, the type of brain injury or trauma to be treated, and the extent and severity thereof.

In another non-limiting embodiment, the at least one inflammation modulatory or anti-inflammatory protein or polypeptide, or biologically active fragment, derivative, or analogue thereof, is administered within 24 hours of the infliction of the brain injury or brain trauma. In yet another non-limiting embodiment, the at least one inflammation modulatory or anti-inflammatory protein or polypeptide, or biologically active fragment, derivative, or analogue thereof is administered within 6 hours of the infliction of the brain injury or brain trauma.

In a further non-limiting embodiment, the at least one inflammation modulatory or anti-inflammatory protein or polypeptide, (e.g., TSG-6), or biologically active fragment, derivative, or analogue thereof is administered at 6 hours after the infliction of the brain injury or brain trauma, and again at 24 hours after the infliction of the brain injury or brain trauma.

In yet another non-limiting embodiment, TSG-6 protein, or a biologically active fragment, derivative, or analogue thereof, is administered in an amount of about 50 μg at 6 hours after the infliction of the brain injury or brain trauma, and against in an amount of about 50 μg at 24 hours after the infliction of the brain injury or brain trauma.

Although the scope of these embodiments is not intended to be limited to any theoretical reasoning, it is believed that when the at least one inflammation modulatory or anti-inflammatory protein or polypeptide, or biologically active fragment, derivative, or analogue thereof, is administered within 24 hours of infliction of the brain injury or brain trauma, that the at least one inflammation modulatory or anti-inflammatory protein or polypeptide (e.g., TSG-6), or biologically active fragment, derivative, or analogue thereof acts as a modulator of the inflammation that results from brain injury or brain trauma, thereby treating or alleviating the adverse effects of the brain injury or brain trauma.

In another non-limiting embodiment, the at least one inflammation modulatory or anti-inflammatory protein or polypeptide, such as TSG-6 or a biologically active fragment, derivative, or analogue thereof, is administered in combination with other therapeutic agents for treating brain injury or brain trauma. Such agents include, but are not limited to, antioxidants, free radical scavengers, ion channel blockers, NMDA antagonists, GABA agonists, and other neuroprotectants that protect neurons from the sequelae of ischemia and hypoxia immediately after injury; anti-apoptotic agents, such as, for example, stanniocalcin-1 (STC-1); antagonists of cardiotonic steroids, such as ouabaine-like factors, marinobufogenins; resibufogein; proteinase inhibitors; inter-alpha-inhibitors; diuretics; and anti-seizure drugs.

In addition, the at least one inflammation modulatory or anti-inflammatory protein or polypeptide, such as TSG-6 or a biologically active fragment, derivative, or analogue thereof, may be administered in combination with one or more coma-inducing drugs in instances where a coma is induced as part of the treatment of the brain injury or trauma.

The invention now will be described with respect to the drawings, wherein:

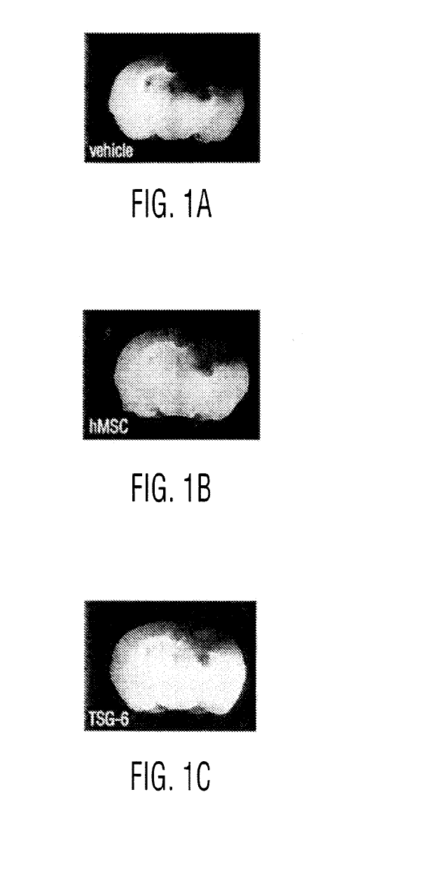

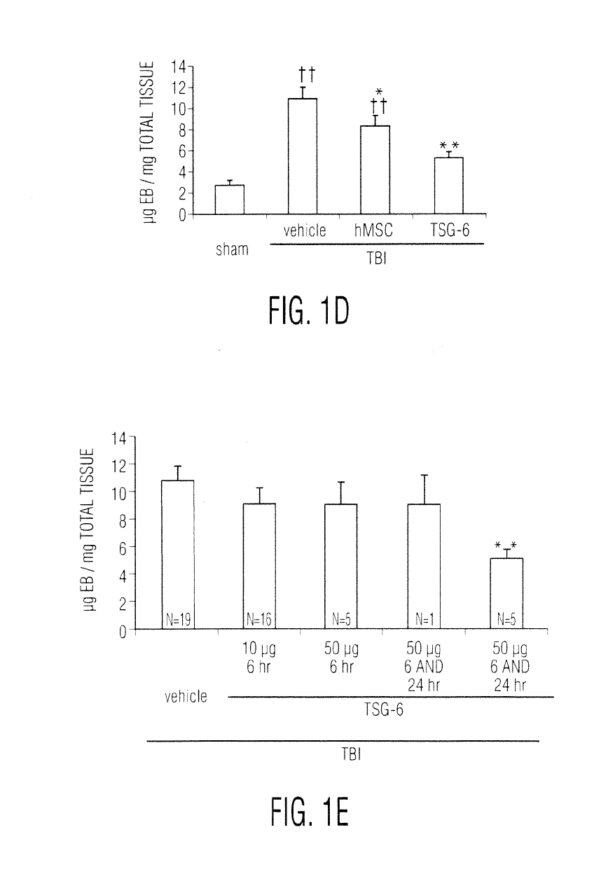

FIG. 1. IV-injected hMSCs or TSG-6 protein decreased BBB permeability in mice with TBI. hMSCs (10⁶ cells/mouse) were administered 6 hr after TBI. TSG-6 protein at dose of 50 μg/mouse was administered twice at 6 and 24 hr after TBI. (A-C) Representative brain slices of the site of cortical contusion injury after administration of vehicle (A), hMSCs (B) or TSG-6 (C) and recovered 3 days post TBI. Blue represents Evans Blue dye extravasation at the site of injury. Scale bars=5 mm (D) Quantitative data of Evans Blue level in the ipsilateral cerebral hemisphere tissue of mice from sham operated group (n=8) and injured group treated vehicle (n=21), hMSCs (n=14) or TSG-6 (n=6). All data are represented s mean±SE. Statistical differences (++p<0.01 from sham, **p<0.01 from vehicle) were determined by one way ANOVA with a Holm multiple comparison test. (E) Dose-dependency and time window of TSG-6 treatment in BBB breakdown following TBI. TSG-6 was administered at 10 or 50 μg/mouse dose once (6 hr after injury) or twice (6 hr and 24 hr after injury). Quantitative data of Evans Blue level in the ipsilateral cerebral hemisphere tissue of mice from injured group treated with vehicle, or TSG-6. Numbers of samples are indicated in the columns. All data are represented as mean±SE. Statistical differences (**p<0.01 from vehicle) were determined by one way ANOVA with a Holm multiple comparison test.

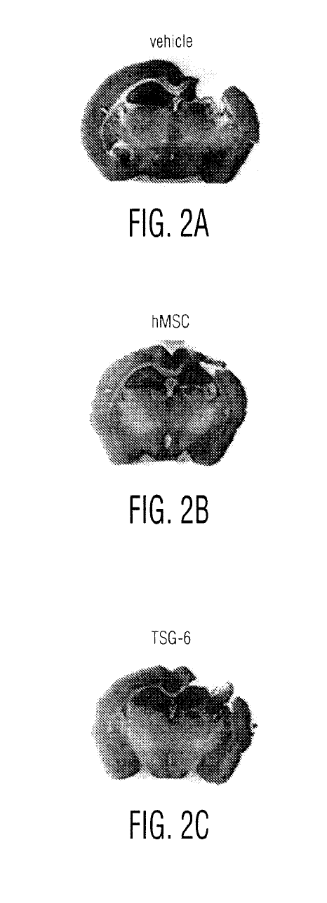

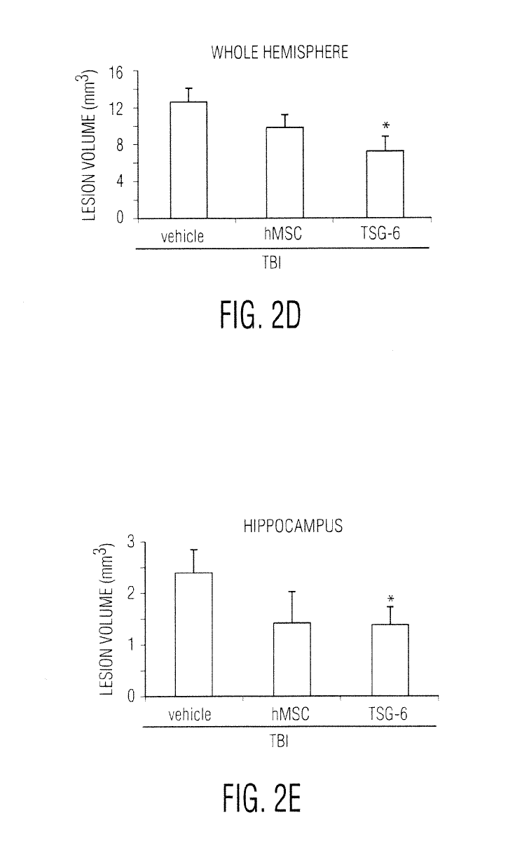

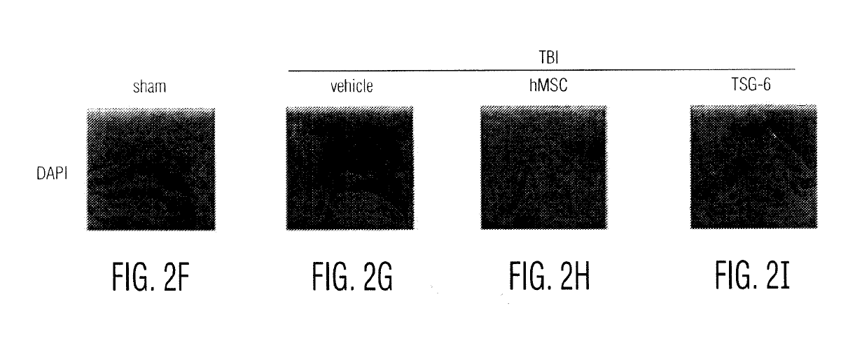

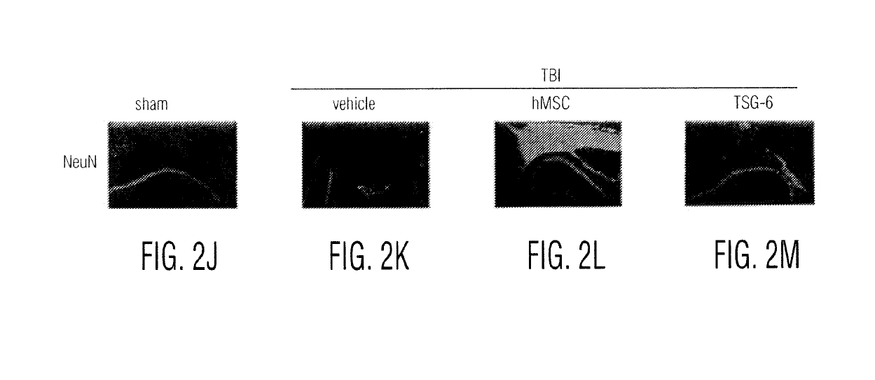

FIG. 2. Intravenous injection of TSG-6 protein protected against TBI induced tissue loss in vivo at 14 days after TBI. hMSCs (10⁶ cells/mouse) were administered 6 hr after TBI. TSG-6 protein at dose of 50 μg/mouse was administered twice at 6 and 24 hr after TBI. (A-C) Representative H&E stained sections from injured mice treated with vehicle (A) MSC (B) or TSG-6 (C) Scale bars=2 mm (D, E) Lesion volume of whole hemisphere (D) and hippocampus (E) All data are represented as mean±SE. n=8 (vehicle), n=5 (hMSC), n=5 (TSG-6). Statistical difference (*p<0.05 from vehicle) were determined by one way ANOVA with a Holm multiple comparison test. (F-I) Representative images of NeuN-stained hippocampus neurons showing neuronal damage (J-M) and counter stained by DAPI (F-I) in mice after TBI. Scale bars=500 μm.

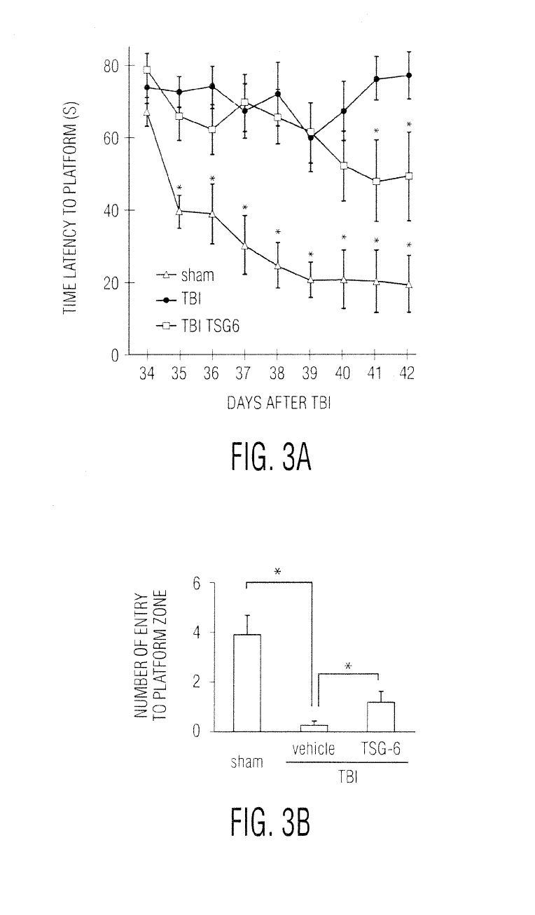

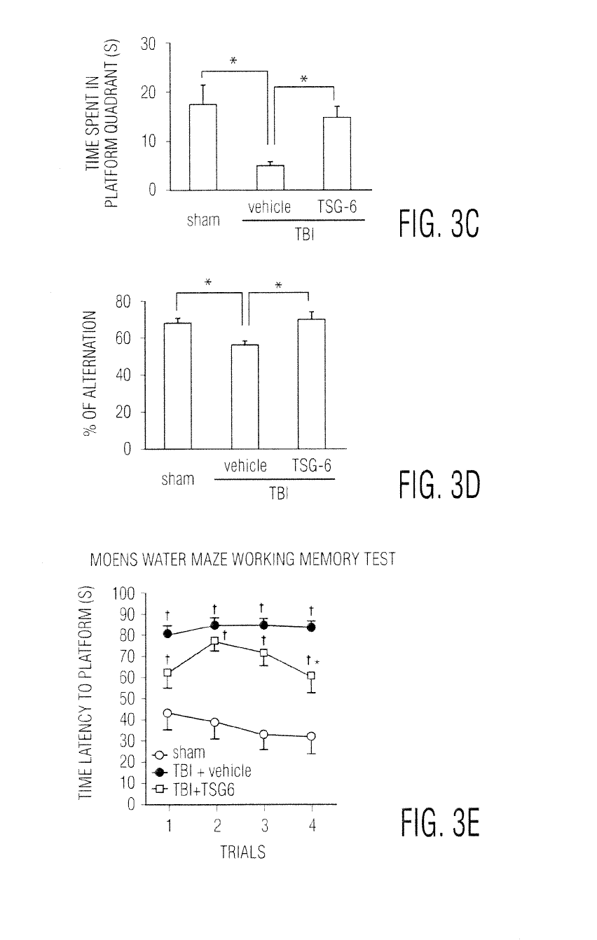

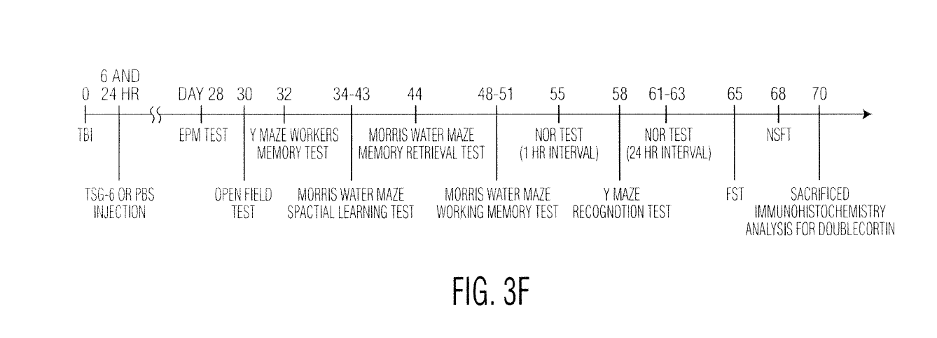

FIG. 3. Protective effect of TSG-6 protein on cognitive function. Effect of TSG-6 on learning and defects in working memory were assessed as latency to locate the hidden platform in Morris water maze (a and e). Probe (memory retention) test was performed at 24 hr. after the last learning session. (b, c) The parameters of the memory retention (number of entry to platform zone, time spent in the platform quadrant) in the TSG-6 treated group are superior to those in the vehicle treated-control group. Injured mice receiving TSG-6 protein showed significant improvement from working memory defects when assessed by Y-maze spontaneous alternation test (d) Assays in (b) and (c) were performed 43 days after TBI. Assay (d) was performed 32 days after TBI. (*p<0.05 from vehicle) (f) Schematic schedule of behavioral tests performed for sham or TBI mice treated with vehicle or TSG-6.

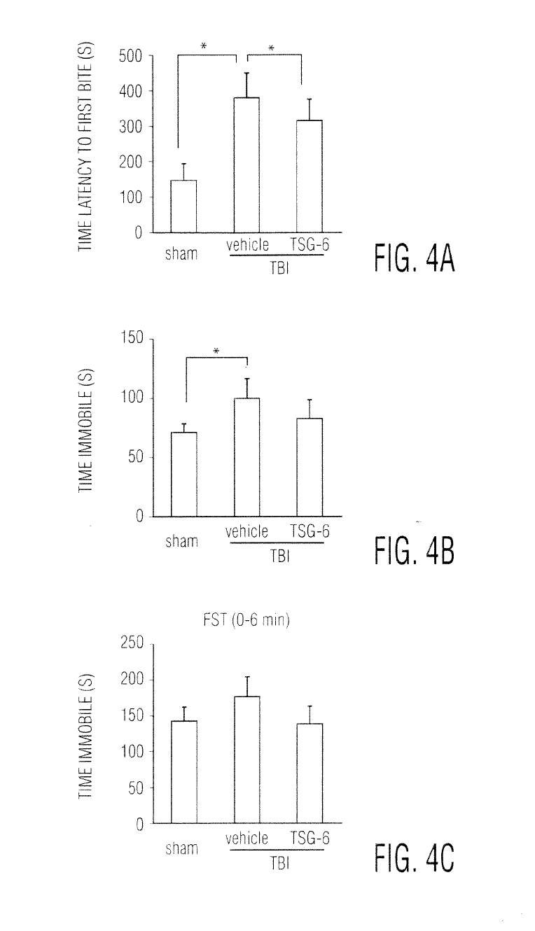

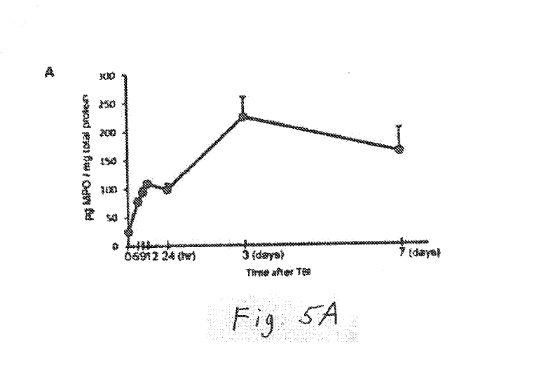

FIG. 4. TSG-6 protein decreases depression-like behavior. Depression-like behavior was assessed by novelty suppressed feeding test (NSFT) (A) and forced swim test (FST). (B and C) The total time spent in immobility for the entire trial duration (C) or last 3 minutes (B) was calculated in the FST. TSG-6 treatment showed statically significant reduction of depressive-like behavior in NSFT. Tendency of antidepressant-like effect of TSG-6 also was observed in FST. Tests in (a), (b) and (c) were performed 68 and 65 days after TBI, respectively. (*p<0.05 from vehicle).

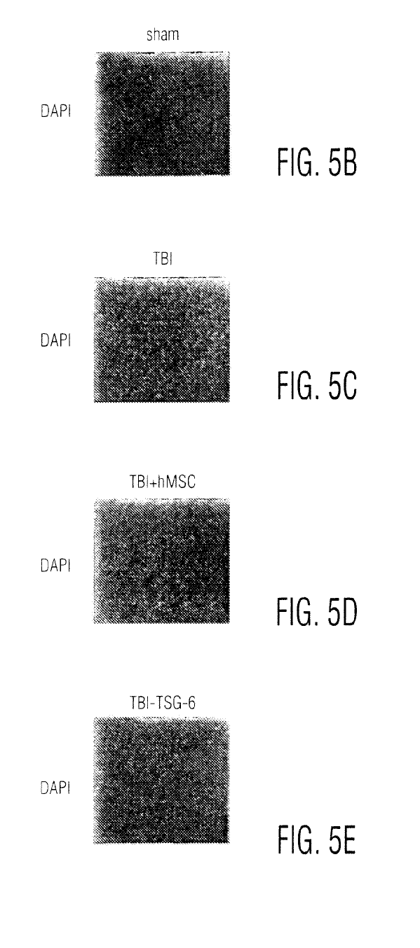

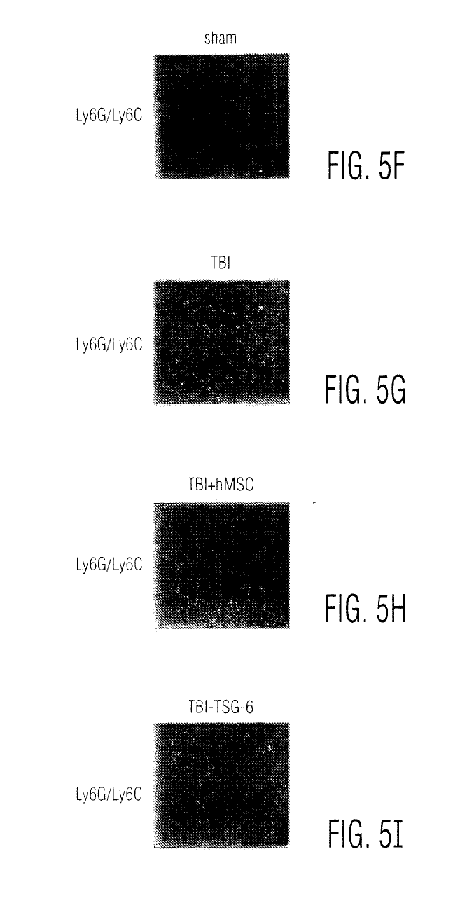

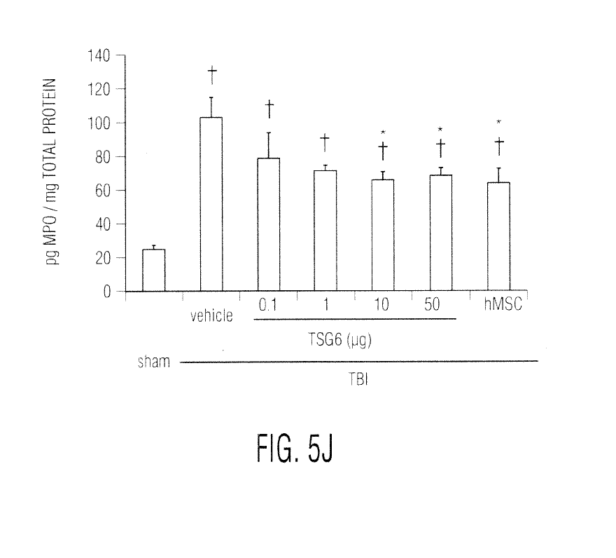

FIG. 5. Reductions in infiltrated neutrophils in mice treated with hMSC or TSG-6 and measured at 24 h after TBI. hMSCs (10⁶ cells/mouse) or TSG-6 protein (50 μg/mouse) were administrated 6 hr after TBI. (A) Time course of neutrophil infiltration as reflected by assays of myeloperoxidase (MPO). n=6 mice/each point. (B-I) Representative images of Ly6G/Ly6C-stained neutrophils infiltration in the cortex from sham operated (F) or injured mouse treated with vehicle (0), hMSC (H) or TSG-6 (I). Sections were counter stained with DAPI (B-E). Scale bars=200 μm. (J) The MPO assayed by ELISA in injured brains of mice treated with vehicle, MSC or indicated dose of TSG-6. n=5 mice/group. All data are represented as mean+SE. Statistical difference (p<0.01, +p<0.05 from sham, *p<0.05 from vehicle) were determined by one way ANOVA with a Holm multiple comparison test.

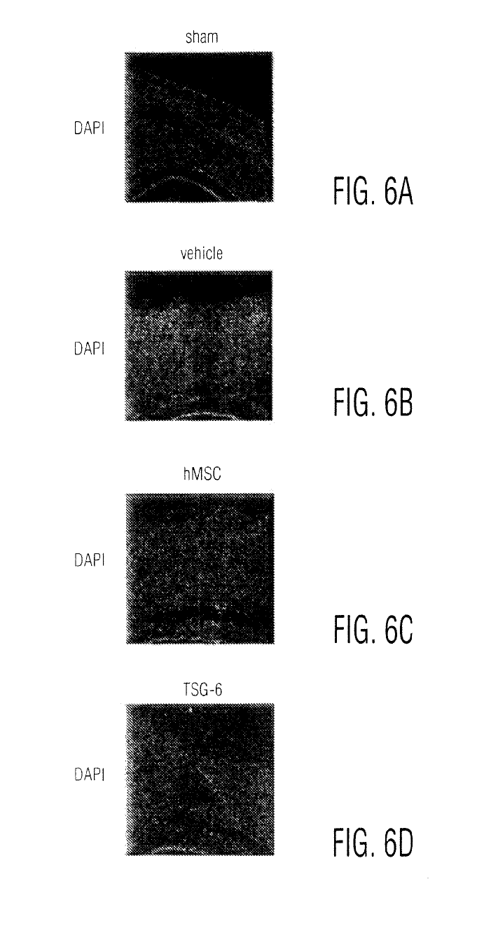

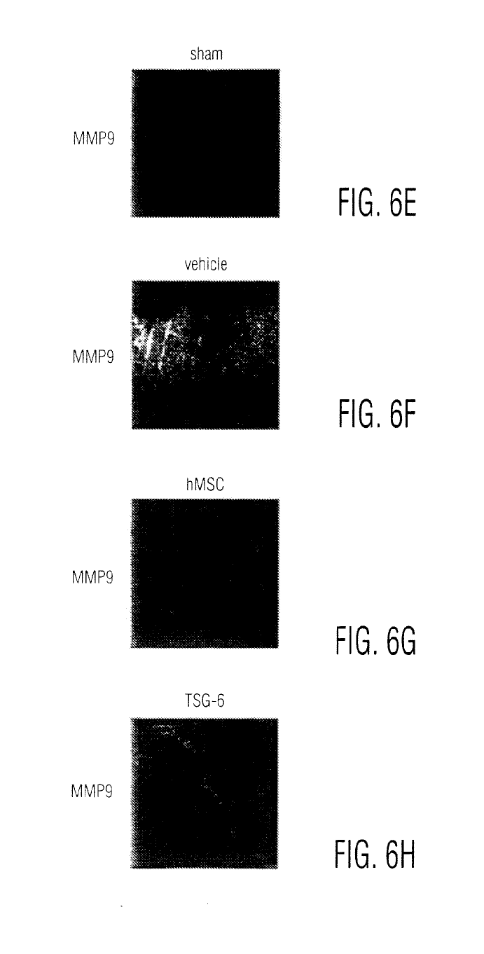

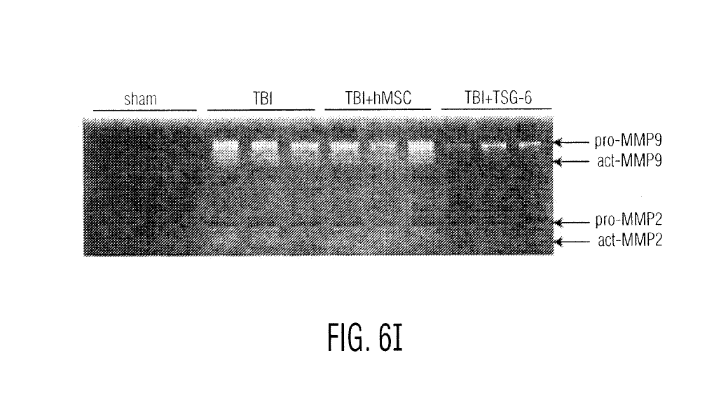

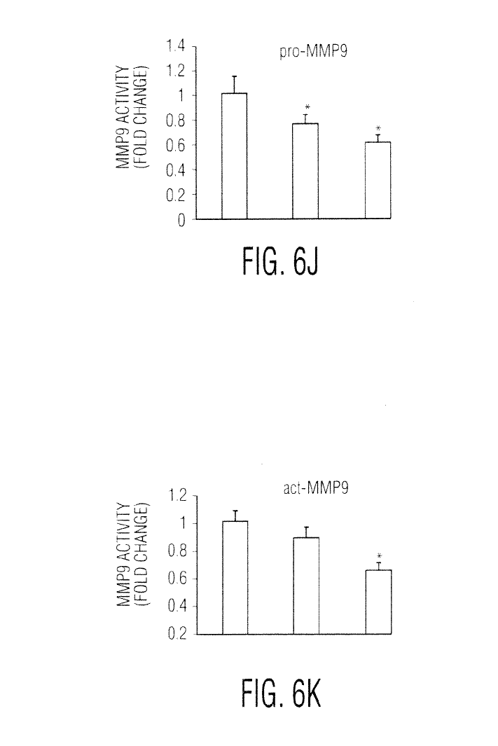

FIG. 6. TSG-6 attenuated TBI-induced expression of matrix metalloproteinase-9 (MMP9) at 24 h after TBI. hMSCs (10⁶ cells/mouse) or TSG-6 protein (50 μg/mouse) were administrated 6 hr after TBI. (A-H) Representative images of MMP-9 immunostained brain from sham operated (E) or injured mouse treated with vehicle (F), hMSC(0) or TSG-6 (H). Counter staining was performed with DAPI (A-D) Scale bars=500 μm. (I) Representative zymogram of MMP9 activity in ipsilateral cortex from sham operated or injured mouse treated with vehicle, hMSC or TSG-6. (J-K) Graphical representation of the average values obtained by densitometric analysis of zymograms for pro-MMP9 (J) or acticvated-MMP9 (K). n=5 mice/group. All data are represented as mean+SE. Statistical difference (*p<0.05 from vehicle) were determined by one way ANOVA with a Holm multiple comparison test.

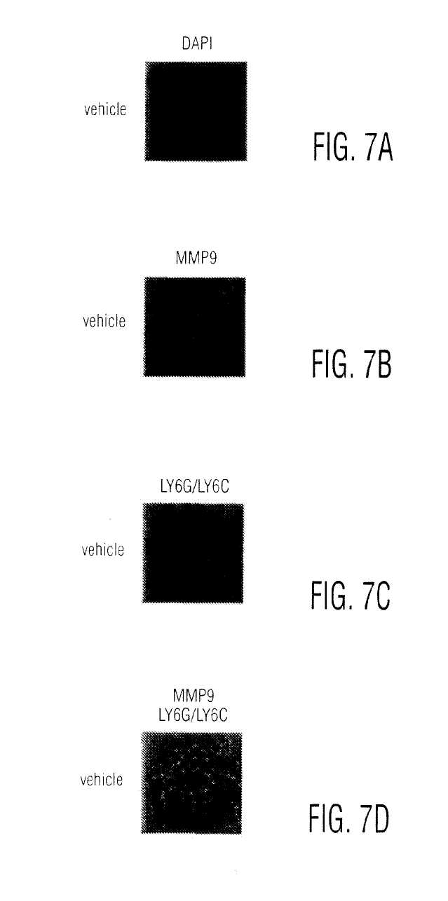

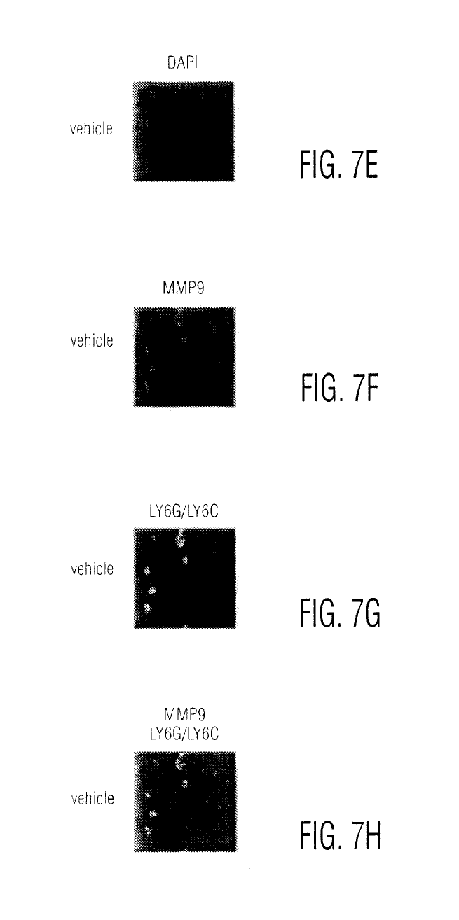

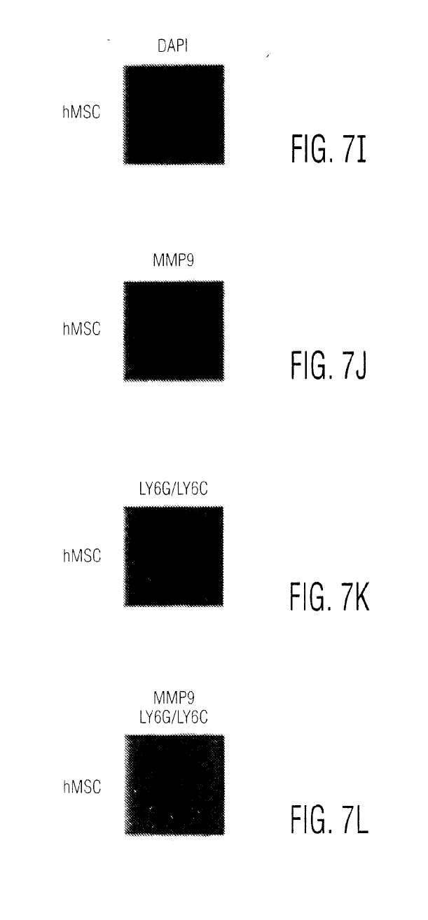

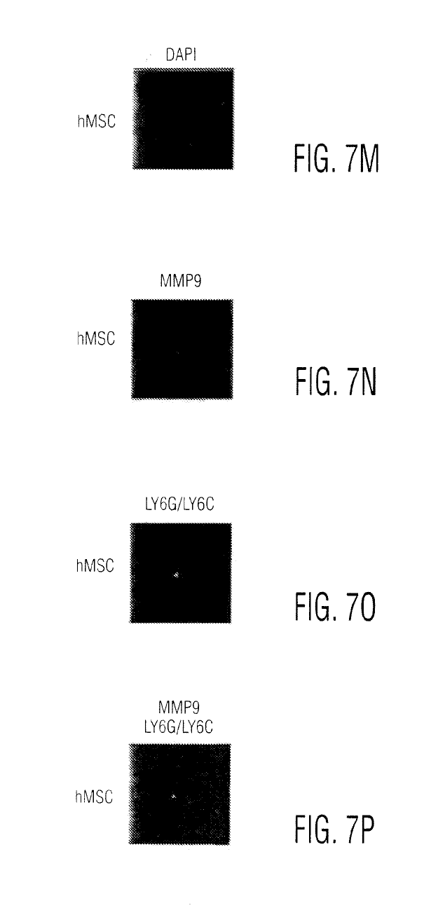

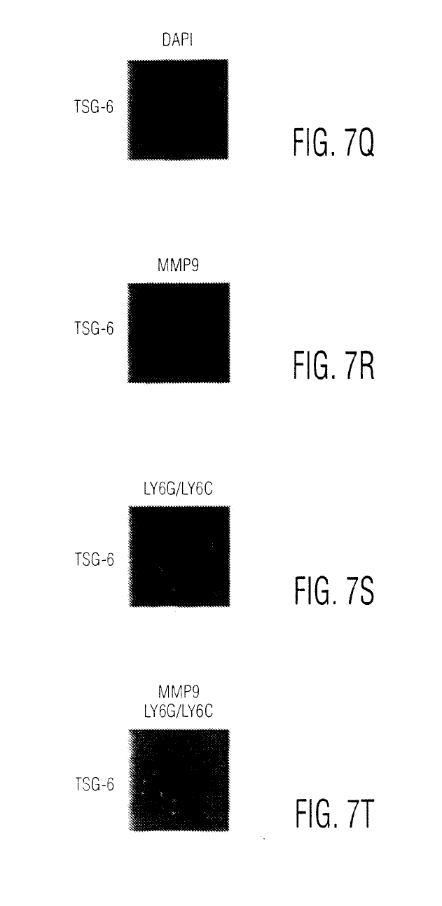

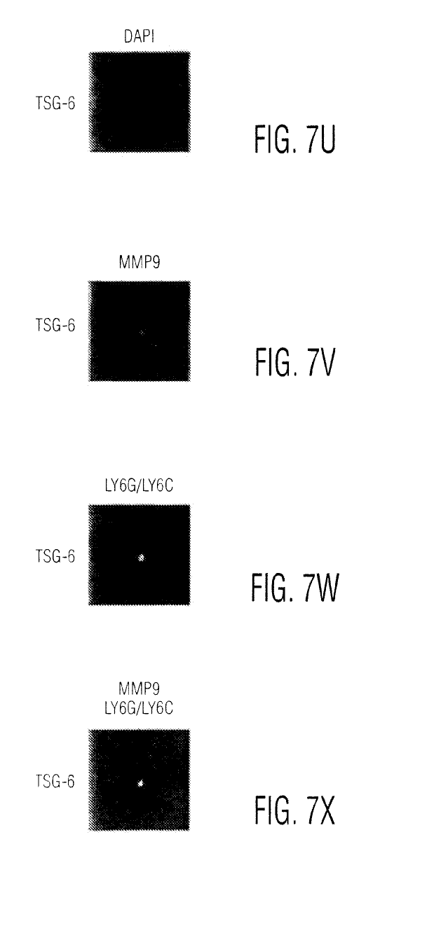

FIG. 7. Neutrophils that infiltrated the brain expressed MMP-9. Representative sections showing double-immunofluorescence labeling of cortical sections from the injured mouse treated with vehicle (A-H), hMSC (I-P) or TSG-6 (Q-X) 24 hr after TBI. hMSCs (10⁶ cells/mouse) orTSG-6 protein (50 μg/mouse) were administrated 6 hr after TBI. Co-labeling of MMP-9 (red) with marker for neutrophils (Ly6G/Ly6C, green). Top: ×20 magnification (scale bars=100 μm). Bottom: ×60 magnification (scale bars=50) μm).

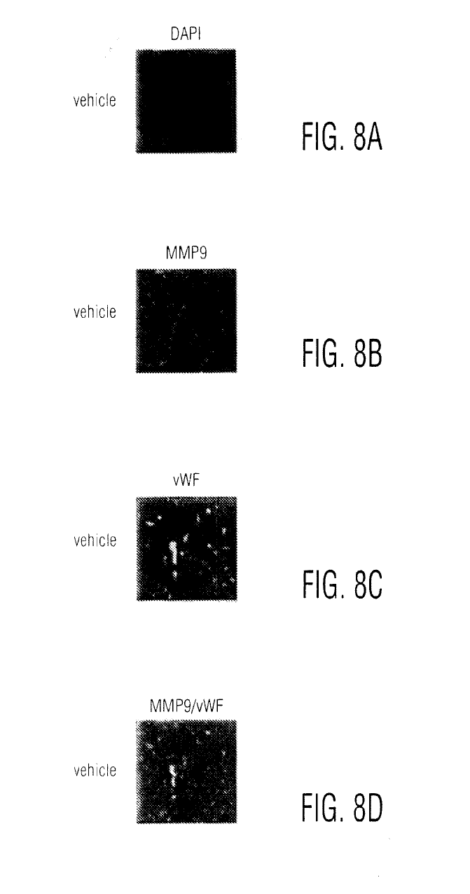

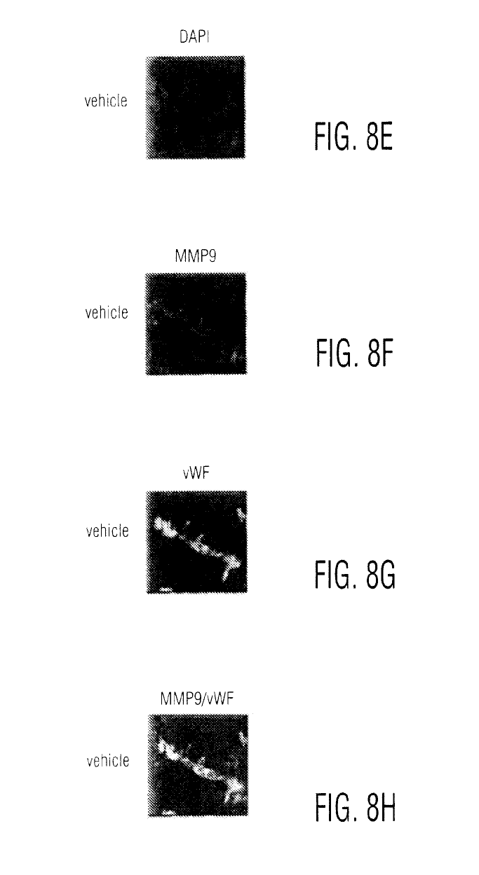

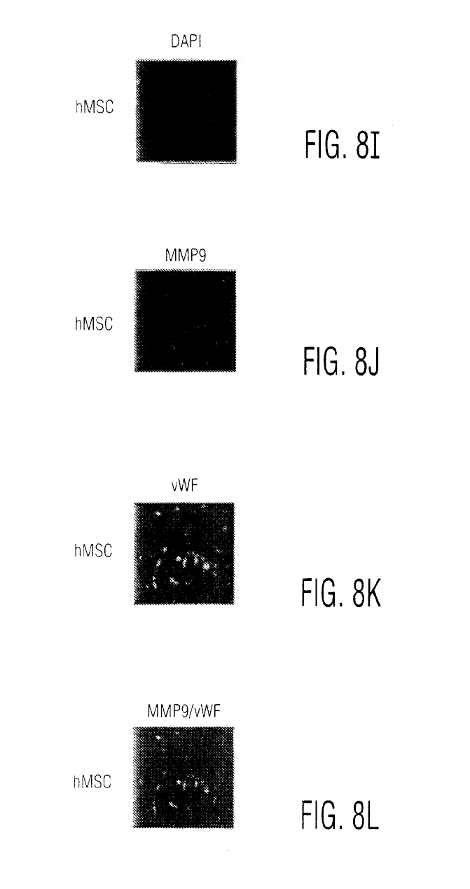

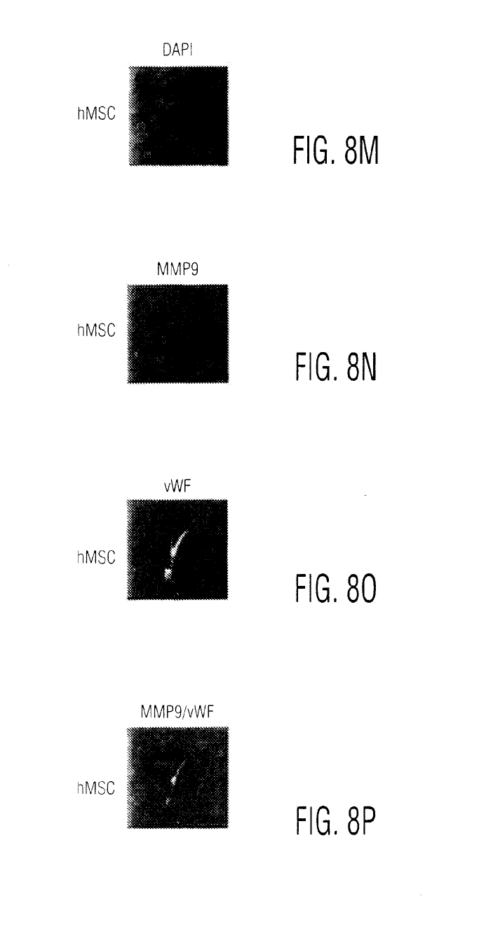

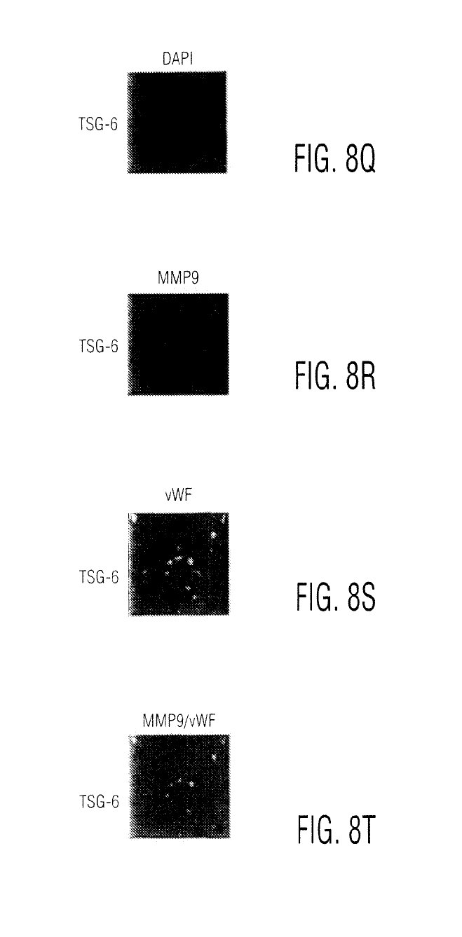

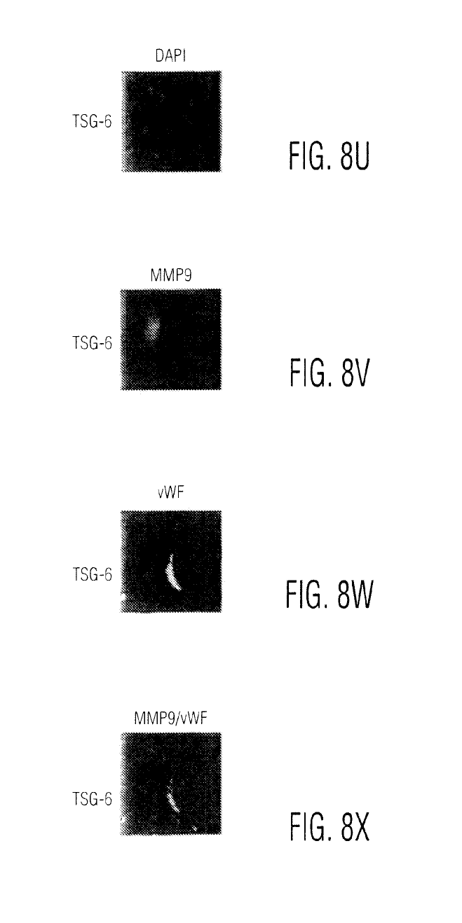

FIG. 8. Blood vessel endothelial cells express MMP-9. Images of representative sections showing double-immunofluorescence detection of cortex sections from the injured mice that received vehicle(A-H), hMSCs (10⁶ cells/mouse); (I-P) or TSG-6 (50 μg/mouse); (Q-X) 24 hr after TBI. Co-labeling of MMP-9 (red) with von Willebrand Factor in blood vessel endothelial cells (vWF, green). Top: ×20 magnification (scale bars=100 μm), Bottom; ×60 magnification (scale bars=50 μm).

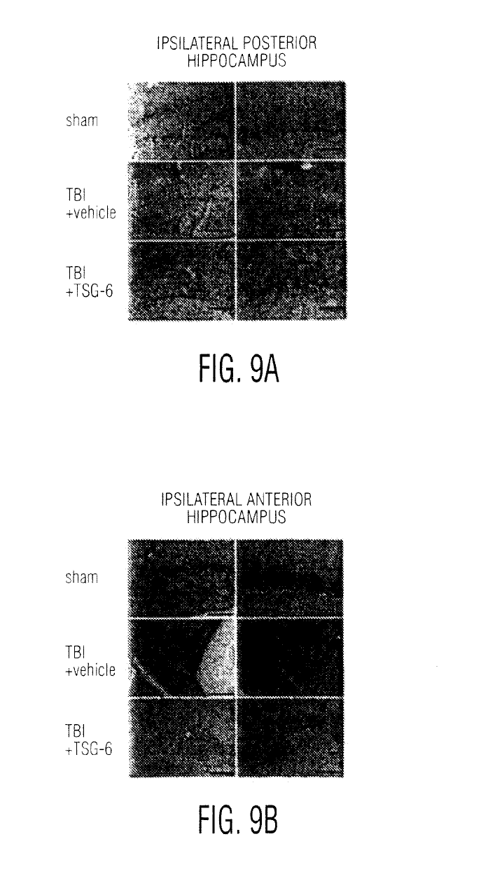

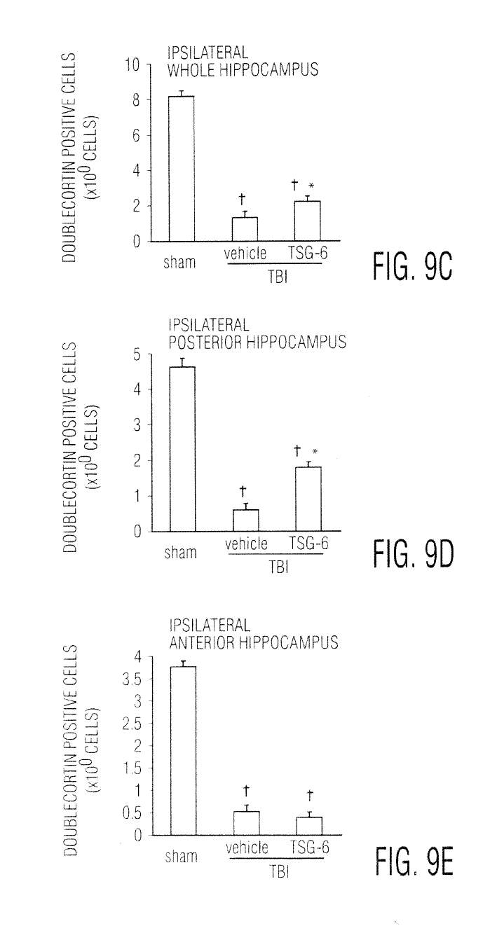

FIG. 9. Administration of TSG-6 protein maintained neurogenesis in the hippocampus. TSG-6 protein (50 μg/mouse) was administered twice at 6 and 24 hr. after TBI. (A,B) Images of representative sections showing distribution of newly born neurons expressing doublecortin (DCX) in the subgranular zone-granule cell layer (SGZ-GCL) of ipsilateral posterior (A) and anterior (B) hippocampus at 10 weeks after TBI from sham and injured mice that received vehicle or TSG-6. Left: ×10 magnification (scale bars=200 μm), Right: ×20 magnification (scale bars=100 μm). (C-E) Numbers of DCX positive newly born neurons. Nine sections were collected from the whole hippocampus, one at each 450 μm. The number of DCX positive neurons was calculated from all nine sections (C), anterior four sections (D) or posterior three sections (E). n=9 or 10 mice/group. All data are represented as mean±SEM. tp,0.05 versus the sham group, *p,0.05 versus the vehicle group.

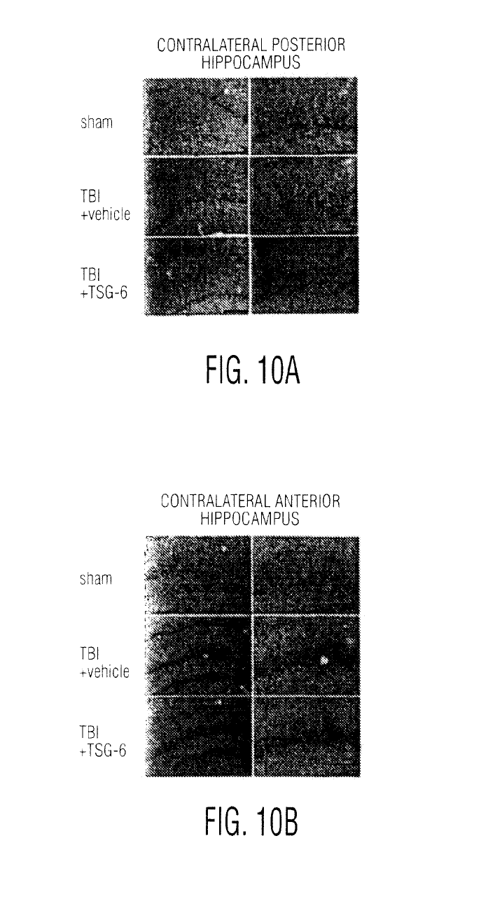

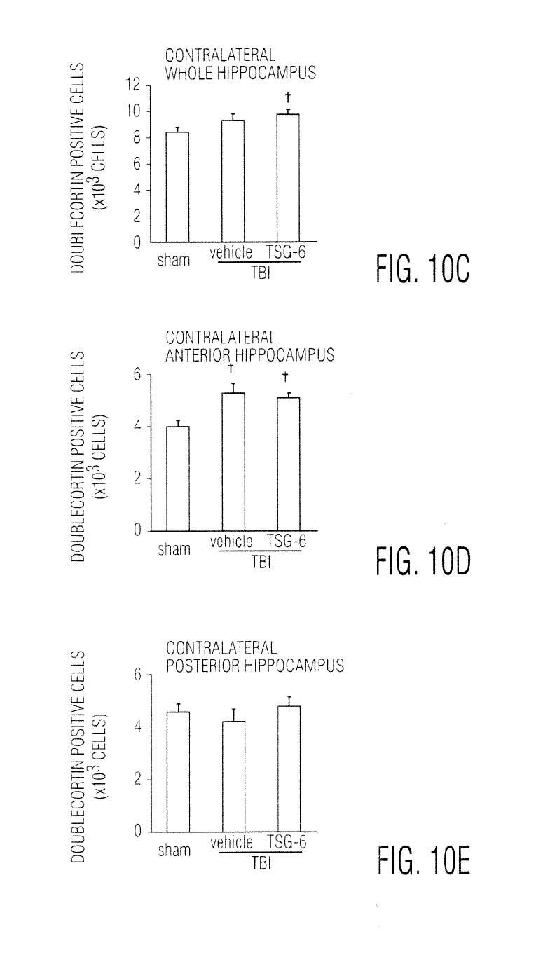

FIG. 10. Administration of TSG-6 protein maintained neurogenesis in the hippocampus. TSG-6 protein (50 μg/mouse) was administered twice at 6 and 24 hr. after TBI. (A,B) Images of representative sections showing distribution of newly born neurons expressing doublecortin (DCX) in the subgranular zone-granule cell layer (SGZ-GCL) of contralateral posterior (A) and anterior (B) hippocampus at 10 weeks after TBI from sham and injured mice that received vehicle or TSG-6. Left:×10 magnification (scale bars=200 μm), Right: ×20 magnification (scale bars=100 μm). (C-E) Numbers of DCX positive newly born neurons. Nine sections were collected from the whole hippocampus, one at each 450 μm. The number of DCX positive neurons was calculated from all nine sections (C), anterior four sections (D) or posterior three sections (E) n=9 or 10 mice/group. All data are represented as mean±SEM. †p,0.05 versus the sham group, *p,0.05 versus the vehicle group.

The invention now will be described with respect to the following example. It is to be understood, however, that the scope of the present invention is not intended to be limited thereby.

### EXAMPLE

### Controlled Cortical Impact Injury (CCI)

Male C57BL/6j mice were purchased from Jackson Laboratories and were 2-3 months old at the time of CCI. All animal experiments were performed in accordance with a protocol approved by the Institutional Animal Care and Use Committee of Texas A&M Health Science Center College of Medicine. A controlled cortical impact device (eCCI Model 6.3; Custom Design and Fabrication at Virginia Commonwealth University Medical Center, Richmond, Va.) was used to administer a unilateral brain injury as described (Mihara et al., 2011). Mice were anesthetized with 4% sevoflurane and O₂ and the head was mounted in a stereotactic frame. The head was held in a horizontal plane, a midline incision was used for exposure, and a 4 mm craniectomy was performed on the right cranial vault. The center of the craniectomy was placed at the midpoint between bregma and lambda, 2 mm lateral to the midline, overlying the tempoparietal cortex. Animals received a single impact with the instrument set to deliver a deformation of 0.8 mm depth with a velocity of 4.5 m/sec and a dwell time of 250 ms using a 3 mm diameter impactor tip. After the injury, a disk made from dental cement was adhered to the skull using Vetbond tissue adhesive (3M, St. Paul, Minn.). The scalp was fastened with sutures. The animal was transferred to a heated recovery cage to be monitored for full recovery from the anesthesia. Sham injured animals were similarly anesthetized and craniectomy performed without cortical injury.

Preparation and Culture of Human MSCs (hMSCs)

hMSCs from normal healthy donors were obtained from the Center for the Preparation and Distribution of Adult Stem Cells (http://medicine.tamhsc.edu/irm/msc-distribution.html). The cells were prepared as previously described (Colter et al., 2000; Sekiya et al., 2002; Wolfe et al., 2008) with protocols approved by an Institutional Review Board of Texas A&M Health Science Center College of Medicine. Frozen vials of passage-1 hMSCs (about 1×10⁶) were thawed, plated on 150 cm² dishes in 20 ml complete MSC medium: α-MEM (GIBCO/BRL, Grand Island, N.Y., USA); 16.6% fetal bovine serum (lot selected for rapid growth; Atlanta Biologicals, Norcross, Ga.); 100 units/mL penicillin (GIBCO/BRL); 100 μg/mL streptomycin (GIBCO/BRL); and 2 mM L-glutamine (GIBCO/BRL), and incubated at 37° C. with 5% humidified CO₂. After 24 hours, the medium was removed and adherent, viable cells were washed with phosphate-buffered saline (PBS), and harvested with 0.25% trypsin/1 mM EDTA (GIBCO/BRL) at 37° C. for 5 min. For expansion, cells were plated at 100 cells/cm² in complete MSC medium and incubated with a medium change every 3-4 days. The cells (passage-2) then were incubated until they reached 70% confluence (approximately 7 days and about 7 population doublings) at which time they were harvested with trypsin/EDTA. The cells were expanded a second time under the same conditions to prepare passage 3 hMSCs that were used for experiments,

### I.V. Infusion of HMSCs and TSG-6

The mice were placed in a tail vein injection restrainer with warming water bath (40° C.) which restrained the animal and gently warmed the tail while allowing access to the tail vein. The hMSCs (10⁶ cells/mouse) or TSG-6 protein (50 μg/mouse, purchased from R&D systems, Minneapolis, Minn.) in a volume of 200 μl PBS were injected using a 27G needle at 6 hr after CCI. Some mice were treated with 50 μg/mouse TSG-6 protein again at 24 hr after CCI. PBS (200 μl) was injected into control mice.

### Evans Blue BBB Permeability Analysis

Evans Blue was used to assess the BBB permeability as this dye has a very high affinity for serum albumin (Rawson, 1942). Seventy two hours after CCI injury, 5% Evans Blue (Sigma-Aldrich, St. Louis, Mo.) in saline was injected via tail vein (4 mL/kg). The dye was allowed to circulate for 2 hr. Animals were anesthetized with a lethal dose of a ketamine/xylazine mix and then perfused transcardially with saline, followed by 4% paraformaldehyde. The brains were harvested and cut into 2 mm sections. After they were photographed, the sections were divided into contralateral and ipsilateral hemispheres. The sections were incubated in 400 μl formamide (Sigma-Aldrich) at 55° C. for 24 h and samples were centrifuged at 20,000 g for 20 min. The supernatant was collected, and the OD at 620 nm was measured using a micro plate reader (BMG LABTECK; Fluostar Omega, Ortenberg, Germany) to determine the amount of Evans Blue in each sample. All values were normalized to hemisphere weight.

### Histological Examination

Mice were anesthetized and perfused transcardiaily with saline and 4% paraformaldehyde. The brains were removed, stored in fresh 4% paraformaldehyde overnight, protected in 20% sucrose, frozen in O.C.T. media (Sakura Finetek, Torrance, Calif.) sectioned (25 μm), and mounted onto slides. Sections were stained with Gill's hematoxylin and eosin (Shandon rapid-chrome frozen section staining kit, Thermo Scientific, Waltham, Mass.) and coverslipped. For volumetric assessment of lesion, images of seven brain sections, taken every 0.5 mm from 0.5 mm to 3.5 mm posterior to Bregma, were captured using a stereomicroscope (SMZ800, Nikon, Melville, N.Y.) and digitalized with an image analysis system (Image J, NIMH, Bethesda, Md.). The area of the lesion in each section was calculated by subtracting the size of ipsilateral cortex from the control contralateral cortex. The lesion volume was computed by integrating the lesion area of each section measured at each coronal level and the distance between two sections (0.5 mm). The volume of the lesion in the hippocampus was measured by same method as described above from five sections taken every 0.5 mm from 1.0 mm to 3.0 mm posterior to Bregma. In addition, sections from 1.5 mm posterior to Bregma were immunostained with anti-NeuN antibody (Table 1) for overnight at 4° C., washed in PBS and incubated with a secondary antibody (Table 1) for 90 min at room temperature. Sections were counterstained with DAPI (Sigma-Aldrich).

### Fluorescence Immunohistochemistry

Twenty four hours after CCI, mice were anesthetized and perfused with PBS and 4% PFA and their brains processed and cut into 12 nm sections described above. The sections were blocked with 5% normal horse serum (NHS, Vector Laboratories, Burlingame, Calif.) and 0.3% Triton-X (Sigma-Aldrich) in PBS (blocking buffer), and incubated with several combinations of primary antibodies (Table 1) in blocking buffer at 4° C. The next day, the sections were washed three times with PBS and incubated with secondary antibodies (Table 1) for 90 min at room temperature. After washing, the sections were counterstained with DAPI for 15 min. Fluorescent images were acquired using a spinning disc fluorescent microscope (Olympus, Center Valley, Pa.) with Slidebook 31 software (Intelligent Imaging Innovations, Denver, Colo.).

TABLE 1

| Antibody | Antigen | Host | Clone | Company | Dilution | Target |
|---|---|---|---|---|---|---|
| NeuN | purified cell | Mouse | A60 | Millipore | 1000 | Neurons |
| | nuclei from | | | | | |
| | mouse brain | | | | | |
| Ly6G/Ly6C | Mouse Ly-6G | Rat | RB6-8C5 | BD | 100 | neutrophils |
| | and Ly-6C | | | Biosciences | | |
| MMP9 | Mouse | Goat | Polyclonal | R&D | 500 | MMP9 |
| | MMP9 | | (AF909) | systems | | |
| vWF | Human von | Rabbit | Polyclonal | Millipore | 50 | Brain |
| | Willebrand | | (AB7356) | | | blood |
| | Factor | | | | | vessels |
| | | | | | | endothelial |
| | | | | | | cells |
| Mouse IgG | Mouse IgG | Goat | | Invitrogen | 500 | Alexa 488 |
| | | | | | | labeled 2^{nd} |
| | | | | | | antibody |
| Rat IgG | Rat IgG | Goat | | Invitrogen | 500 | Alexa 488 |
| | | | | | | labeled 2^{nd} |
| | | | | | | antibody |
| Goat IgG | Goat IgG | Donkey | | Invitrogen | 500 | Alexa 592 |
| | | | | | | labeled 2^{nd} |
| | | | | | | antibody |
| Rabbit IgG | Rabbit IgG | Goat | | Invitrogen | 500 | Alexa 488 |
| | | | | | | labeled 2^{nd} |
| | | | | | | antibody |
| DCX | Human | Goat | C18 | Santo Cruz | 250 | Newborn |
| | Doublecortin | | (sc-8066) | | | neurons |
| Goat IgG | Goat Ig G | Horse | | Vector Labs | 200 | Biotin |
| | | | | | | labeled |
| | | | | | | 2nd |
| | | | | | | antibody |

### ELISA for Myeloperoxidase (MPO)

For protein extraction, the injured brain hemisphere was homogenized with disperser (T10; IKA Wilmington, N.C.) in lysis buffer containing 200 mM NaCl, 5 mM EDTA, 10 mM Tris-HCl (pH 7.4), 10% glycerin, 1 mM PMSF and protease inhibitor cocktail (Thermo Scientific). The samples were sonicated on ice and centrifuged twice (15,000×g at 4 ″C for 20 min). The supernatant was assayed for protein with the BradFord reagent (Ameresco, Solon, Ohio), and for myeloperoxidase by ELISA (MPO ELISA kit; HyCult Biotech, Plymouth Meeting, Pa.).

### Zymograms

Mice were killed at 24 h post-CCI. The brains were removed rapidly, and damaged brain tissue within the traumatized hemisphere was homogenized in lysis buffer containing 50 mmol/L Tris-HCl (pH 8.0), 150 mmol/L NaCl, 1% IMP-40, 0.5% deoxycholate, and 0.1% SDS. Soluble and insoluble extracts were separated by centrifugation (20,000 g, 30 min at 4° C.). After the protein concentration was measured by BradFord reagent (Ameresco), samples containing 20 μg total protein were analyzed by gel zymography using precast gelatin gels (10% Zymogram Gelatin Gels; Invitrogen/Novex). With constant gentle agitation, gels were renatured in Novex Zymogram Renaturing buffer (Invitrogen/Novex) for 30 minutes at room temperature, developed in Novex Zymogram Developing Buffer (Invitrogen/Novex) overnight at 37° C., stained with Colloidal Blue (Invitrogen/Novex), and washed extensively with distilled water (>20 hours) to yield uniform background signal. Digital images of stained wet gels were captured using a scanner. The images were analyzed with the densitometry using Image J software.

### Behavior Tests

Behavior tests were performed as described previously (Parihar et al., 2011). An experimental time-line is shown in FIG. 3F. Elevated plus maze test and open field test were performed to assess anxiety-like behavior. Morris water maze test, Y maze spontaneous alternation test and novel object recognition test were performed to evaluate memory function. Depression-like behavior was analyzed by forced swim test and novelty suppressed feeding test. Details on each behavior test are described hereinbelow.

### Morris Water Maze Test

Mice underwent learning and memory testing during the daylight period. The water maze tank (a circular plastic pool measuring 120 cm in diameter and 60 cm in height) was filled with 30° C. water containing milk to a 30 cm height and extra-maze cues were placed on the walls of the room. Before training periods, mice were allowed to swim for 45 seconds in the pool without platform in order to become familiar with swimming. Swim speed and total travel distance were calculated in this time. Mice were trained first to find the circular platform (10 cm in diameter) submerged in water within one of the 4 quadrants using spatial cues. The movement of mice in the water maze was videotaped continuously and recorded using the computerized ANY-Maze video-tracking system. The training comprised 9 sessions over 9 days with 4 acquisition trials per session. Each trial lasted 90 seconds and the inter-trial interval was 60 seconds. During different trials, the mouse was placed in the water facing the wall of the pool in a pseudo-random manner so that each trial commenced from a different start location. Once the mouse reached the platform, it was allowed to stay there for 15 seconds. When a mouse failed to find the platform within the ceiling period of 90 seconds, it was guided into the platform where it stayed for 15 seconds. The location of the platform remained constant across all days and trials for an individual animal. After each trial, the mouse was wiped thoroughly with dry towels, air dried and placed in the home cage. During the 9-day acquisition period, the latency to reach the platform was measured as an indicator of learning ability. The latency to find the platform was recorded for every trial. From these, the mean latency to reach the platform in every session was calculated. One day after the above 9-day learning paradigm, mice were subjected to a 45 second retention (probe) test. For this, the platform was removed and the mice were released from the quadrant opposite to the original position of the platform. Number of entries into the platform area and dwell time in the platform quadrant was measured. Typically, mice that are capable of retrieving the learned memory easily head straight to the platform area after release, spend most of the trial (45 sec) searching within the quadrant (or area) where the platform was placed originally and exhibit many platform area crossings. Thus, mice exhibiting increased numbers of platform area entries and greater dwell time in the platform area are considered to have superior memory than mice exhibiting fewer platform area entries, greater latency to reach the platform area, and reduced dwell time in the platform area.

The procedures described above are for the assessment of trial-independent learning (that is, the goal does not move from trial to trial during a given phase of testing). To assess working memory, a different method was performed. In this procedure, the platform was relocated every day and the animal was given four trials per day. On each day, the first trial represented a sample trial. During the sample trial, the animal must learn the new location of the platform by trial-and-error. Trials 2 through 4 began after a 15 second inter-trial interval. The latency to find the platform was recorded for every trial. From these, the mean latency to reach the platform in each trial for 4 days was calculated. If the animal recalls the sample trial, it swims a shorter path to the goal on the following trial. As the platform was moved daily, no learning of platform position from the previous day could be transferred to the next day's problem; hence, recall on each day during Trials 3 and 4 was dependent on that day's sample trial and measured only working memory.

Y-maze test

Y maze spontaneous alternation is a behavioral test for measuring the willingness of rodents to explore new environments. The first session measures working memory in mice by scoring the number of alternations which the mouse does in Y-maze when the animal visits all three arms without going into same arm twice in a row. The experimental apparatus consisted of Y-shaped maze with three gray opaque plastic arms at a 120° angle from each other. ANY-maze video tracking system was used to record and analyze the animal's movement within the maze. After introduction to the center of the maze, the animal was allowed to explore the three arms freely for 5 min. Over the course of multiple arm entries, the subject should show a tendency to enter a less recently visited arm. The number of arm entries and the number of triads was recorded in order to calculate the percentage of alternation.

The second session includes two trials. During trial 1, one of the arms of the maze was blocked, allowing for a 5 min exploration of only two arms of the maze. After a 30 min delay, trial 2 was started. During trial 2, all three arms were available for another 5 min exploration. Trial 2 takes advantage of the innate tendency of mice to explore novel unexplored areas (e.g., the previously blocked arm). The time spent in novel unexplored areas of each animal was measured. Mice with intact recognition memory prefer to explore a novel arm over the familiar arms, whereas mice with impaired spatial memory enter all arms randomly. Thus, trial 2 represents a classic test for spatial recognition memory.

### Novelty Suppressed Feeding Test (NSFT)

All mice were subjected to fasting for twenty four hours before the commencement of the test but water was provided ad libitum. During the test, food pellets (regular chow) was placed on a circular piece of white filter paper positioned in the center of an open field (45×45 cm) that was filled with approximately 2 cm of animal bedding. Each mouse was removed from its home cage and placed in a corner of the open field. The test lasted for 10 minutes. The latency to the first bite of the food pellet was recorded (defined as the mouse sitting on its haunches and biting the pellet with the use of its forepaws). It is well known that latency to the first bite is much shorter in normal mice than depressed mice. The overall latency to the first bite determines the extent of depressive-like behavior in individual mice.

### Forced Swim Test (FST)

Each mouse was first placed in a glass beaker (having an inner diameter of 10 cm and depth of 15 cm) filled with tap water (−25° C.) to a depth of 10 cm. The depth of water used ensured that the animal could not touch the bottom of the container with their hind paws. The FST was conducted in a single session comprising 6 minutes and data were collected every minute for swimming, climbing (or struggling) and immobility (or floating) during the procedure. Swimming in the FST is defined as the horizontal movement of the animal in the swim chamber and climbing refers to the vertically directed movement with forepaws mostly above the water along the wall of the swim chamber. On the other hand, immobility or floating is defined as the minimum movement necessary to keep the head above the water level. Mice were removed from the water at the end of 6 minutes and gently dried and placed back in their home cages. From the collected data, the total time spent in immobility for the trial duration was calculated for every mouse and utilized as an index of depressive-like behavior.

### Doublecortin (DCX) Immmohistochemistry

Serial sections (every fifteenth) through the entire hippocampus were selected in each animal belonging to different groups and processed for DCX immunostaining using a goat polyclonal antibody to DCX (Table 1) using the ABC method, as detailed in a previous study (Rao et al., 2005).

### Quantification of the Numbers of Newly Born Neurons (DCX Positive Neurons) in the Hippocampus.

In order to determine the status of hippocampal neurogenesis, stereological quantifications of DCX positive cells in the SGZ-GCL were performed using serial, total nine sections (every fifteenth) immunostained for DCX. Total number of DCX positive cells was calculated by integrating the number of DCX positive cells on each section multiplied by 15.

### Statistical Analysis

All data are represented as mean±SE. One way ANOVA with a Holm multiple comparison test was carried out with JSTAT software to determine statistically significant differences for all data. P<0.05 was considered to be significant.

### Results

Intravenous Administration of TSG-6 Protein Decreased BBB Permeability in Mice 3 Days after TBI

To test whether TSG-6 treatment of TBI mice decreases BBB leakage, we measured the extravasation of Evans Blue dye into the brain. FIG. 1A to C show that intravenous administration of either hMSC or TSG-6 significantly decreased BBB leakage on day 3 compared with control TBI mice as assayed by leakage of albuimin-bound Evans Blue into the parenchyma of the brain. The concentration of albumin-Evans Blue in brain extracts from TSG-6 administrated mice was decreased by 51.6% (p<0.05) and to a level that was not statistically different from the values from sham operated mice (FIG. 1D). A single administration of 10⁶ hMSCs at 6 hours after TBI was effective. In order to study a dose response and time window to the therapy, 10 or 50 μg/mouse doses of TSG-6 were injected once (6 hr after injury) or twice (6 hr and 24 hr after injury). A significant decrease in BBB permeability was observed only when 50 μg/mouse of TSG-6 protein was administered twice (FIG. 1E).

### TSG-6 Treatment Reduced Lesion Size in TBI Mice

Two weeks after cortical contusion injury, TBI was found to induce a lesion (including cavity) which was extensive, spreading from the cortex through the hippocampus and connecting to the lateral ventricles (FIG. 2A). Treatment with hMSCs or TSG-6 tended to reduce the size of the lesion, but TSG-6 appeared to be more effective. (FIGS. 2B and 2C). Quantitative analyses showed that total lesion volume in the whole hemisphere was reduced by 40% following TSG-6-administration (FIG. 2D, P<0.05). In the hippocampus, injured control mice lost 2.40±0.44 cm³ of tissue, compared to 1.45±0.33 cm³ for injured mice that received TSG-6 (FIG. 2E, P<0.05). Administration of hMSCs tended to reduce the volume of the lesion but the difference from control was not statistically significant (FIG. 2 A to E).

To investigate the constitution of neuronal cell layers in the hippocampus after injury, we stained neurons with NeuN, a marker for mature neurons. CA1 and CA3 pyramidal cell layers of control TBI mice were destroyed entirely (FIG. 2K). hMSC or TSG-6 treatment attenuated this damage significantly (FIGS. 2L and 2M).

Improved Cognitive Function at 6 to 7 wk after TBI.

We also tested whether treatment with TSG-6 during the Phase I of inflammation had a long-term effect on cognitive function. As expected, assays in the Morris water maze (See experimental schedule in FIG. 3F) demonstrated that TBI in the mice caused severe defects in spatial learning (FIG. 3A). Mice treated with TSG-6 during the first 24 hr after TBI demonstrated better learning ability. Also, the TSG-6 treated mice successfully retrieved memory in the probe test (FIGS. 3B and C) and the working memory test in the Morris water maze (FIG. 3E). In addition, they demonstrated improvement in the Y-maze working memory test (FIG. 3D).

Decreased Depressive-Like Behavior at 9 Weeks after TBI

In addition, we tested the mice for depressive-like behavior 9 weeks after TBI. The treatment with TSG-6 within the first 24 hours of TBI improved results in the novelty suppressed feeding test (NSFT; FIG. 4A). Furthermore, in the last 3 min of forced swim test (FST), control TBI mice exhibited increased immobility (or floating) behavior (FIG. 4B). TSG-6 treatment after TBI reduced this depressive-like behavior to levels seen in sham control mice (FIG. 4B). Similar trend was seen when floating behavior was assessed for the entire duration of FST (FIG. 4C). Thus, TSG-6 treatment after TBI considerably reduces depressive-like behavior, which is an indication of improved mood function or antidepressive-like effect mediated by TSG-6.

Effects of TSG-6 on Inflammation after TBI

To explore the mode of action of TSG-6, we examined the extent of inflammation after TBI. Immunohistochemistry staining against a neutrophil marker (Ly6G/Ly6C) of the cortical sections from control injured mice demonstrated extensive infiltration of neutrophils at 24 hr following an injury (FIG. 5G). There was significantly less neutrophil infiltration in the cortex of mice that received TSG-6 (FIG. 5I). For a quantitative measure of neutrophil infiltration, the ipsilateral cortexes were assayed for the myeloperoxidase (MPO) concentration. Treatment with TSG-6 decreased the levels of MPO by 34% (FIG. 5B, p<0.05) in the brain. To study a dose response to the therapy, varying doses of TSG-6 (0.1-50 μg/mouse) were injected via tail vein. The statistically significant decreases of MPO expression levels were observed from administration of 10 and 50 μg/mouse of TSG-6 (FIG. 5J). Although it was not statistically significant, there was a trend of decreasing MPO expression after administration of 0.1 and 1 μg/mouse.

### TSG-6 Suppressed MMP9 Activity Following TBI

Previous studies showed leukocyte-derived MMP9 mediated BBB breakdown after focal cerebral ischemia (Gidday et al, 2005) and MMP-9 contributed to the pathophysiology of traumatic brain injury (Wang et al., 2000). In addition, our group reported TSG-6 protein suppressed MMP9 activity in rodent models of myocardial infarction and chemical injury of the cornea (Lee et al., 2009; Oh et al., 2010). To test whether TSG-6 treatment of TBI mice decreases MMP9 protein expression and activity, we performed immunohistostaining and zymography for MMP9. Following TBI, a high level of MMP9 expression was observed in the entire damaged cortex (FIG. 6E) compared to sham injured cortex (FIG. 6D). In contrast, there was marked reduction in the level of MMP9 expression in the brains of TSG-6 treated mice (FIG. 6H). Gel zymography confirmed the observations in that TSG-6 treatment suppressed the activities of pro-MMP9 and activated-MMP9 by 43% and 37%, respectively (FIGS. 6 J and K. p<0.05). The injured cortex was also assayed for cells expressing MMP9. After TBI, numerous cells in the injured cortex were immunoreactive for MMP9 (FIGS. 6F, 7B and 8B). The Ly6G/Ly6C immunopositive neutrophils were co-localized with MMP9 (FIG. 7 A-H). Ly6G/Ly6C and MMP9 double-immunopositive cells still were observed in brains of TSG-6 treated mice, but the number was decreased dramatically (FIG. 7Q-X). Strong MMP9 immunoreactivity was also detected in vWF immunopositive-brain blood vessels endothelial cells (FIG. 8A-H). The MMP9 expression in endothelial cells also declined significantly after TSG-6 treatment (FIG. 8 Q-X).

Increased Neurogenesis in the Hippocampus at 10 Weeks after TBI.

Some of the cognitive deficits associated with inflammation may be related to decreased neurogenesis in the hippocampus (Kohman and Rhodes, 2013). Therefore, we performed immunostaining for doublecortin (DCX), a marker of newly born neurons, of brains obtained 10 weeks after TBI. In hippocampus ipsilateral to TBI, DCX positive newly born neurons in the subgranular zone-granule cell layer were decreased by 85% after TBI (FIGS. 9A and C). TSG-6 treatment increased the newly born neurons by 1.7-fold (P<0.05). This protective effect of TSG-6 was apparent in the posterior region of the hippocampus (2.4 fold increase compared to vehicle treated group, P<0.05. FIG. 9D). In the anterior region of the hippocampus, the structure of the hippocampus was preserved better in the TSG-6 treated group (FIG. 9B), but the number of DCX positive neurons was comparable to control TBI mice (FIG. 9E). These data suggest that TSG-6 modulated inflammation in the peripheral damaged area. Interestingly, DCX positive newly born neurons in the contralateral side of the hippocampus also were increased in TBI mice receiving TSG-6 (FIGS. 10A and C). The results suggested that TSG-6 improved cognitive and mood function at least in part by up-regulation of hippocampal neurogenesis.

### DISCUSSION

The results here demonstrated that intravenous injection of TSG-6 protected the brain from BBB breakdown and decreased the volume of the lesion produced by TBI. Moreover, damage to the hippocampal CA1 and CA3 pyramidal neurons was decreased significantly. In addition, administration of TSG-6 greatly suppressed neutrophil infiltration and MMP-9 activity after the injury.

TSG-6 protein, a hyaluronan-binding protein comprised mainly of a Link and CUB module arranged in a contiguous fashion (Milner and Day, 2003), has been shown previously to be a potent inhibitor of neutrophil migration in an in vivo model of acute inflammation (Wisniewski et al., 1996). Also transgenic mice with null alleles for TSG-6 demonstrated enhanced neutrophil extravasation when challenged with proteoglycan-induced arthritis (Szanto et al., 2004). The protein has several modes of action. One effect is to interrupt the inflammatory cascade of proteases by binding to inter-α-inhibitor and enhancing its inhibitory activity (Mahoney et al., 2005). Another effect is to bind to and thereby inactivate pro-inflammatory fragments of hyaluronan; however, some of the anti-inflammatory activity was shown to be independent of its ability to bind HA or to potentiate the inhibitory activity of inter-α-inhibitor (Getting et al., 2002). Also, TSG-6 was reported to modulate the adhesion of neutrophils to the endothelium (Cao et al., 2004). In addition, our research group found that TSG-6 decreased zymosan/TLR2/NFκ-B signaling in resident macrophages and thereby modulated the initial phase of inflammatory responses (Choi et al., 2011). Similar results were obtained in a rodent model of chemical injury to the cornea (Oh et al., 2010). Of special interest was that in these models TSG-6 acted during the small initial phase of the inflammatory response and thereby decreased the large secondary phase that is counteracted by most anti-inflammatory agents. Also of interest was that TSG-6 exerted similar neutrophil inhibitory effects in different models of inflammation and regardless of whether it is administered intravenously or directly into a site of inflammation (Wisniewski et al., 1996; Getting et al., 2002; Lee et al., 2009; Oh et al., 2010; Roddy et al., 2011). It thus seems likely that TSG-6 acts via the circulation to influence a fundamental process of neutrophil recruitment and extravasation. We observed here that the neutrophil extravasation into the brain clearly was decreased in mice treated with TSG-6 intravenously 6 hr after TBI (FIG. 5). At this time point, the breakdown of the BBB is not maximal (Zhao et al., 2007). Therefore, our data suggested that the primary effect of the TSG-6 was to reduce inflammation, apparently by its systemic action.

The inflammatory response in patients with TBI begins within hours after injury and lasts up to several weeks (Morganti-Kossmann et al., 2007). Animal models of TBI have shown that an influx of peripheral neutrophils occurs following injury, with a time course that correlates with BBB disruption (Ghajar, 2000). Macrophages, natural killer cells, T helper cells, and T cytotoxic-suppressor cells are also present in the brain following TBI (Holmin et al., 1998). Following infiltration, leukocytes release pro-inflammatory cytokines, cytotoxic proteases and reactive oxygen species. The factors released from leukocytes further mediate the recruitment of hematopoietic cells from the periphery, perpetuate activation of resident CNS cells, and contribute to the overall increase in BBB permeability (Shlosberg et al., 2010). Therefore, the timely resolution of leukocyte extravasation is essential to prevent damage to healthy tissue. Previous study showed that neutrophil depletion reduces BBB breakdown, axon injury, and inflammation after intracerebral hemorrhage (Moxon-Emre and Schlichter, 2011). Here, we demonstrated that TSG-6 treatment significantly decreased neutrophil infiltration into injured brain (FIG. 5) and BBB disruption (FIG. 1). These therapeutic effects of TSG-6 may have contributed to a decreased damage observed in the cerebrum and hippocampus (FIG. 2).

Furthermore, we demonstrated that TSG-6 treatment suppressed MMP-9 activity (FIG. 6) expressed by neutrophils (FIG. 7) and endothelial cells of brain blood vessels (FIG. 8). MMPs comprise a family of zinc endopeptidases that can modify several components of the extracellular matrix (Yong et al., 2001). In particular, the gelatinases MMP-2 and MMP-9 can degrade the neurovascular matrix. Following TBI, activation and up-regulation of MMPs, which degrade the neurovascular basal lamina, lead to a further increase in blood vessel permeability and, as a result, contribute to the development of edema (Suehiro et al., 2004). MMP-9 knockout mice have reduced BBB leakage and infarction volume after cerebral ischemia (Asahi et al., 2001). Neutrophils provide the main source of MMPs in TBI and the other brain diseases (Cuzner and Opdenakker, 1999; Vlodaysky et al., 2006). Our data suggests that TSG-6 reduces MMP-9 activity via suppression of neutrophil infiltration. On the other hand, Cheng et al., (2006) observed that activated protein C, which is a plasma serine protease with systemic anticoagulant, anti-inflammatory, and antiapoptotic activities, inhibits a pro-hemorrhagic tissue plasminogen activator-induced, NF-KB-dependent matrix metalloproteinase-9 pathway in ischemic brain endothelium. These observations suggest that TSG-6 also can suppress MMP9 activity via its ability to increase the plasmin-inhibitory activity of inter-α-inhibitor. This suggestion can explain our data that TSG-6 treatment suppresses the MMP9 expression in cerebral blood vessel endothelial cells as well as neutrophils (FIG. 8). Further investigation into how TSG-6 regulates MMP9 production in TBI will be of interest in future studies.

There have been multiple attempts to use anti-inflammatory agents in animal models and clinical trials of TBI. Essentially all have failed (Ransohoff and Brown, 2012; Rivest, 2011). The approach here to test TSG-6 in TBI is novel in two respects. One is that the therapy was administered acutely and only during the first 24 hr. following TBI. Therefore, the therapy was targeted to a time when inflammation is more likely to be harmful than helpful. The second novelty in the approach is that the protein employed does not fit the usual definition of an anti-inflammatory agent: in response to acute tissue injury in both the cornea (Oh et al., 2010) and the peritoneum (Choi et al., 2011), it acted during the initial Phase I of the inflammatory response by binding directly to or through hyaluronan to CD44 in a manner that modulated TLR2/NF-k B signaling in resident macrophages, which are the sentinel cells in most tissues that receive the initial signals of damage associated molecular patterns (DAMPs) (Medzhitov, 2010). In both cornea and peritonitis models, TSG-6 did not inhibit inflammation completely. In the corneal model in which the time window was explored (Oh et al., 2010), it had little if any effect when administered after 6 hr. and at the onset of the large Phase II of the inflammatory response (Oh et al., 2010). Therefore TSG-6 probably is classified better as an inflammation modulatory protein than an anti-inflammatory agent.

The results demonstrated that the initial mild Phase I of the inflammatory response following TBI is more protracted than in other tissues and persisted for at least 24 hr. In effect, a longer time is required in the brain for the normal sequence of events in inflammation, i.e. the time required for sensor cells (macrophages, dendritic cells, and mast cells in peripheral tissues) to respond to DAMPs from injured cells and to release mediators (cytokines, chemokines, bioactive amines, ecasonoids, and proteolytic products such as bradykinin) that usher in the massive edema and invasion of neutrophils, macrophages, and lymphocytes that characterize inflammation (Medzhitov, 2010). The longer time for Phase I suggested that the time window for therapy with TSG-6 might be as long as 24 hr. Intravenous administration of either human MSCs or TSG-6 about 6 hr. after TBI were effective about equally in decreasing the inflammatory response in terms of neutrophil infiltration and the level of MMP9 activity in endothelial cells and invading neutrophils at 24 hr. Two administrations of TSG-6, one at 6 and one at 24 hr., however, were more effective than a single administration of either hMSCs or TSG-6 in BBB maintenance at day 3 and in preserving neural tissue 2 weeks after the TBI. Most importantly, the two administrations of TSG-6 during the first 24 hr after TBI improved memory, depressive-like behavior, and neurogenesis in the hippocampus after 6 weeks.

The results reported here are consistent with previous observations on TBI. An influx of peripheral neutrophils occurs following TBI, with a time course that correlates with BBB disruption (Ghajar, 2000). Timely resolution of leukocyte extravasation is essential to reduce damage to healthy tissue. A previous study showed that neutrophil depletion reduced BBB breakdown, axon injury, and inflammation after intracerebral hemorrhage (Moxon-Emre and Schlichter, 2011). Our results showed that TSG-6 treatment after TBI decreased neutrophil infiltration significantly as well as BBB permeability. These observations demonstrate that early TSG-6 administration after TBI can modulate inflammation.

Common consequences of TBI include personality changes, cognitive problems and a reduced quality of life calling for long-term rehabilitation and treatment (Masel and DeWitt, 2010). The mechanisms underlying TBI-induced cognitive and behavioral impairments are unclear. Neuroinflammation recently was reported to decrease neurogenesis and impair aspects of cognitive function (Russo et al., 2011). On the other hand, activation of more chronic inflammatory pathways was reported to be important for regenerative responses (Schmidt et al., 2005). The inflammatory process thus presents both negative and positive consequences to the post-injury process (Rivest, 2011). Acute administration of TSG-6 rescued both tissue damage and neurogenesis. Interestingly, TSG-6 also up-regulated neurogenesis in hippocampus contralateral to injury as long as 10 weeks after TBI.

The mechanism whereby TSG-6 modulated inflammation in the TBI model may or may not be the same as its mechanism of action in peripheral tissues. Macrophages are not present in the central nervous system; their function is sub-served largely by microglia and in part by astrocytes. Microglia express TLRs and TLRs in the brain and the genes were up-regulated by TBI (Hua et al., 2011). Therefore microglia, or specific subset of microglia, may respond to TSG-6 in a manner similar to resident macrophages in other tissues.

Alternatively, TSG-6 may be acting primarily on the monocytes/macrophages that invade the brain as the blood brain barrier is disrupted by TBI. Also, some of the many other actions of TSG-6 may be involved. The protein was discovered as cDNA clone number 6 and was isolated after cultures of fibroblasts were stimulated with TNF-α (Wisniewski and Vilcek, 2004). It was shown subsequently to be expressed by a variety of cells in response to stimulation by pro-inflammatory cytokines (FOldp et al., 1997; Milner et al., 2006; Milner and Day, 2003; Szanto et al., 2004; Wisniewski and Vilcek, 2004). TSG-6 can stabilize the extracellular matrix and thereby limit the invasion of inflammatory cells by binding to hyaluronan, heparin, heparin sulfate, thrombospondins-1 and −2, and fibronectin (Baranova et al., 2011; Blundell et al., 2005; Kuznetsova et al., 2005; Kuznetsova, et al., 2008; Mahoney et al., 2005). In addition, it can inhibit the cascade of proteases released by inflammation by its complex catalytic interaction with inter-α-inhibitor (Rugg et al., 2005; Scavenius et al., 2011; Zhang et al., 2012), or by forming ternary complexes with mast cell trypases and heparin (Nagyeri et al., 2011). In apparently independent interactions, TSG-6 also reduces the migration of neutrophils through endothelial cells (Cao et al., 2004), and inhibits FGF-2 induced angiogenesis through an interaction with pentraxin (Leali et al., 2012). It is not clear which of these effects may be involved in suppressing inflammation after TBI. TSG-6 remains an attractive therapeutic agent, in part because no toxicities were reported in the many experiments performed in rodents with recombinant TSG-6 (Milner et al., 2006; Wisniewski and Vilcek, 2004).

Here we provided evidence that acute treatment with TSG-6 is highly effective not only in decreasing the initial injury to the brain but also in decreasing the long-term memory and behavioral disabilities observed in a mouse model for TBI. The results therefore suggest that acute administration of TSG-6 is potentially an attractive therapy for patients with TBI.

Optimization of the management and prevention of secondary damage following TBI poses a notable challenge to the medical community. Currently, no readily available neuroprotective agent exists that can prevent effectively or reverse the damage caused by secondary delayed pathologies following TBI. Here we provide novel evidence that TSG-6 treatment is highly efficacious for prevention of secondary neural damage. For that reason, TSG-6 could be an ideal neuroprotective compound for reducing brain damage and dysfunction after TBI.

The disclosures of all patents, publications (including published patent applications), depository accession numbers, and database accession numbers are incorporated herein by reference to the same extent as if each patent, publication, depository accession number, and database accession number were incorporated individually by reference.

It is to be understood, however, that the scope of the present invention is not to be limited to the specific embodiments described above. The invention may be practiced other than as particularly described and still be within the scope of the accompanying claims.

### REFERENCES

Asahi M, Wang X, Mori T, Sumii T, Jung J C, Moskowitz M A, et al. Effects of matrix metalloproteinase-9 gene knock-out on the proteolysis of blood-brain barrier and white matter components after cerebral ischemia. J Neurosci 2001; 21: 7724-32.
Baranova, et al. The inflammation-associated protein TSG-6 cross-links hyaluronan via hyaluronan-induced TSG-6 oligomers. J. Biol. Chem. 2011; 286:25675-25686.
Blundell, et al. Towards a structure for a TSG-6 hyaluronan complex by modeling and NMR spectroscopy: insights into other members of the link module superfamily. J. Biol. Chem. 2005; 280:18189-18201.
Browne, et al. Chronic ibuprofen administration worsens cognitive outcome following traumatic brain injury in rats. Exp. Neurol 2006; 201:301-307.
Cao T V, La M, Getting S J, Day A J, Perretti M. Inhibitory effects of TSG-6 Link module on leukocyte-endothelial cell interactions in vitro and in vivo. Microcirculation 2004; 11: 615-24.
Chen, et al. Glucocorticoids aggravate retrograde memory deficiency associated with traumatic brain injury in rats. Neurotrauma. 2009; 26:253-260.
Cheng T, Petraglia A L, Li Z, Thiyagarajan M, Zhong Z, Wu Z, et al. Activated protein C inhibits tissue plasminogen activator-induced brain hemorrhage. Nat Med 2006; 12:1278-85.
Choi H, Lee R H, Bazhanov N, Oh J Y, Prockop D J. Anti-inflammatory protein TSG-6 secreted by activated MSCs attenuates zymosan-induced mouse peritonitis by decreasing TLR2/NF-kappaB signaling in resident macrophages. Blood 2011; 118: 330-8.
Chopp M, Li Y, Zhang J. Plasticity and remodeling of brain. J Neural Sci 2008; 265: 97-101.
Colter D C, Class R, DiGirolamo C M, Prockop D J. Rapid expansion of recycling stem cells in cultures of plastic-adherent cells from human bone marrow. Proc Natl Acad Sci USA 2000; 97: 3213-8.
Coronado V G, Xu L, Basavaraju S V, McGuire L C, Wald M M, Faul M D, et al. Surveillance for traumatic brain injury-related deaths-United States, 1997-2007. MMWR Surveill Summ 2011; 60: 1-32.
Cuzner M L, Opdenakker G. Plasminogen activators and matrix metalloproteases, mediators of extracellular proteolysis in inflammatory demyelination of the central nervous system. J Neuroimmunol 1999; 94:1-14.
Edwards, et al. Final results of MRC CRASH, a randomized placebo-controlled trial of intravenous corticosteroid in adults with head-injury outcomes at 6 months. Lancet 2005; 365:1957-1959.
Flach, A. Topically applied nonsteroidal anti-inflammatory drugs and corneal problems: interim review and comment. Ophthalmology. 2000; 107:1224-1226.
Foraker J E, Oh J Y, Ylostalo J H, Lee R H, Watanabe J, Prockop D J. Cross-talk between human mesenchymal stem/progenitor cells (MSCs) and rat hippocampal slices in LPS-stimulated cocultures: the MSCs are activated to secrete prostaglandin E2. J Neurochem 2012; 119: 1052-63.
Fülöp, et al. Coding sequence, exon-intron structure and chromosomal localization of murine TNF-stimulated gene 6 that is specifically expressed by expanding cumulus cell-oocyte complexes. Gene 1997; 202:95-102.
Getting S J, Mahoney D J, Cao T, Rugg M S, Fries E, Milner C M, et al. The link module from human TSG-6 inhibits neutrophil migration in a hyaluronan- and inter-alpha-inhibitor-independent manner. J Biol Chem 2002; 277: 51068-76.
Ghajar J. Traumatic brain injury. Lancet 2000; 356: 923-9.
Gidday J M, Gasche Y G, Copin J C, Shah A R, Perez R S, Shapiro S D, et al.
Leukocyte-derived matrix metalloproteinase-9 mediates blood-brain barrier breakdown and is proinflammatory after transient focal cerebral ischemia. Am J Physiol Heart Circ Physiol 2005; 289: H558-68.
Harting M T, Jimenez F, Xue H, Fischer U M, Baumgartner J, Dash P K, et al. Intravenous mesenchymal stem cell therapy for traumatic brain injury. J Neurosurg 2009; 110:1189-97.
Holmin S, Soderlund J, Biberfeld P, Mathiesen T. Intracerebral inflammation after human brain contusion. Neurosurgery 1998; 42: 291-8; discussion 8-9.
Hua, et al. Genomic profile of Toll-like receptor pathways in traumatically brain-injured mice: effect of exogenous progesterone. Journal of Neuroinflammation 2011; 8:42.
Kohman, R., Rhodes, J. Neurogenesis, inflammation and behavior. Brain Behay. Immun. 2013; 27:22-32.
Kopen G C, Prockop D J, Phinney D G. Marrow stromal cells migrate throughout forebrain and cerebellum, and they differentiate into astrocytes after injection into neonatal mouse brains. Proc Natl Acad Sci USA 1999; 96:10711-6.
Kovesdi, et al. Acute minocycline treatment mitigates the symptoms of mild blast-induced traumatic brain injury. Frontiers in Neurology. 2012; 3:111.
Kuznetsova, et al. The N-terminal module of thrombospondin-1 interacts with the link domain of TSG-6 and enhances its covalent association with the heavy chains of inter-alpha-trypsin inhibitor. J. Biol. Chem. 2005; 280:30899-30908.
Kuznetsova, et al. TSG-6 binds via its CUB-C domain to the cell-binding domain of fibronectin and increases fibronectin matrinx assembly. Matrix Biology 2008; 27:201-210.
Leali, et al. Long pentraxin 3/tumor necrosis factor-stimulated gene-6 interaction: a biological rheostat for fibroblast growth factor 2-mediated angiogenesis. Arterioscler. Throm. Vasc. Biol. 2012; 32:696-703.
Lee R H, Pulin A A, Seo M J, Kota D J, Ylostalo J, Larson B L, et al. Intravenous hMSCs improve myocardial infarction in mice because cells embolized in lung are activated to secrete the anti-inflammatory protein TSG-6. Cell Stem Cell 2009; 5: 54-63.
Lin, et al. Corneal melting associated with use of topical nonsteroidal anti-inflammatory drugs after ocular surgery. Arch. Opthalmol. 2000; 118:1129-1132.
Maas, et al., Moderate and severe traumatic brain injury in adults. Lancet Neurology 2008; 7: 728-741.
Mahmood A, Lu D, Chopp M. Intravenous administration of marrow stromal cells (MSCs) increases the expression of growth factors in rat brain after traumatic brain injury. J Neurotrauma 2004; 21: 33-9.
Mahoney D J, Mulloy B, Forster M J, Blundell C D, Fries E, Milner C M, et al. Characterization of the interaction between tumor necrosis factor-stimulated gene-6 and heparin: implications for the inhibition of plasmin in extracellular matrix microenvironments. J Biol Chem 2005; 280: 27044-55.
Masel, B., DeWitt, D. Traumatic brain injury: a disease process, not an event. J. Neurotrauma 2010; 27:1529-1540.
Medzhitov, R. Inflammation 2010: new adventure of an old flame. Cell 2010; 140:771-776.
Mihara Y, Dohi K, Yofu S, Nakamachi T, Ohtaki H, Shioda S, et al. Expression and localization of the orexin-1 receptor (OX1R) after traumatic brain injury in mice. J Mol Neurosci 2011; 43:162-8.
Milner, et al. TSG6: a pluripotent inflammatory mediator? Biochem. Soc. Trans. 2006; 34:446-450.
Milner C M, Day A J. TSG-6: a multifunctional protein associated with inflammation. J Cell Sci 2003; 116:1863-73.
Morganti-Kossmann M C, Satgunaseelan L, Bye N, Kossmann T. Modulation of immune response by head injury. Injury 2007; 38:1392-400.
Moxon-Emre I, Schlichter L C. Neutrophil depletion reduces blood-brain barrier breakdown, axon injury, and inflammation after intracerebral hemorrhage. J Neuropathol Exp Neurol 2011; 70: 218-35.
Munoz J R, Stoutenger B R, Robinson A P, Spees J L, Prockop D J. Human stem/progenitor cells from bone marrow promote neurogenesis of endogenous neural stem cells in the hippocampus of mice. Proc Natl Acad Sci USA 2005; 102:18171-6.
Myburgh, et al., Epidemiology and 12-month outcomes from traumatic brain injury in Australia and New Zealand. J. Trauma. 2008; 64: 854-862
Nagyeri, et al. TSG-6 protein, a negative regulator of inflammatory arthritis, forms a ternary complex with murine mast cell tryptases and heparin. J. Biol. Chem. 2011; 286:23559-23569.
Oh J Y, Roddy G W, Choi H, Lee R H, Ylostalo J H, Rosa R H, Jr., et al. Anti-inflammatory protein TSG-6 reduces inflammatory damage to the cornea following chemical and mechanical injury. Proc Natl Acad Sci USA 2010; 107:16875-80.
Ohtaki H, Ylostalo J H, Foraker J E, Robinson A P, Reger R L, Shioda S, et al. Stem/progenitor cells from bone marrow decrease neuronal death in global ischemic by modulation of inflammatory/immune responses. Proc Natl Acad Sci USA 2008; 105:14638-43.
Parr A M, Tator C H, Keating A. Bone marrow-derived mesenchymal stromal cells for the repair of central nervous system injury. Bone Marrow Transplant 2007; 40: 609-19.
Pati S, Khakoo A Y, Zhao J, Jimenez F, Gerber M H, Harting M, et al. Human mesenchymal stem cells inhibit vascular permeability by modulating vascular endothelial cadherin/beta-catenin signaling. Stem Cells Dev 2011; 20: 89-101.
Prockop D J. “Stemness” does not explain the repair of many tissues by mesenchymal stem/multipotent stromal cells (MSCs). Clin Pharmacal Ther 2007; 82: 241-3.
Ransohoff, R Brown, M. Innate immunity in the central nervous system. J. Clin. Invest. 2012; 122:1164-1171.
Rao, et al. Newly born cells in the aging dentate gyrus display migration, survival, and neural fate choice but enforce retarded early maturation. The European Journal of Neuroscience. 2005; 21-464-476.
Rawson R H. The binding of T-1824 and structurally related diazo dyes by the plasma proteins. American Journal of Physiology 1942; 138: 708-17.
Rivest, S. The promise of anti-inflammatory therapies for CNS injuries and diseases. Expert Review of Nerotherapeutics. 2011; 11:783-786.
Roberts, et al. Effect of intravenous corticosteroids on death within 14 days in 10008 adults with clinically significant head injury (MRC CRASH trial): randomized placebo-controlled trial. Lancet 2004; 364:1321-1328.
Roddy G W, Oh J Y, Lee R H, Bartosh T J, Ylostalo J, Coble K, et al. Action at a distance: systemically administered adult stem/progenitor cells (MSCs) reduce inflammatory damage to the cornea without engraftment and primarily by secretion of TNF-alpha stimulated gene/protein 6. Stem Cells 2011; 29:1572-9.
Rugg, et al. Characterization of complexes formed between TSG-6 and inter-alpha-inhibitor that act as intermediates in the covalent transfer of heavy chains onto hyaluronan. J. Biol. Chem. 2005; 280: 25674-25686.
Russo, J., et al. Effects of neuroinflammation on the regenerative capacity of brain stem cells. J. Neurochem. 2011; 116:947-956.
Scavenius, et al. Human inter-α-inhibitor is a substrate for Factor XIIIa and tissue transgultaminase. Biochem. Biophys. Acta. 2011; 1814:1624-1630.
Schrepfer S, Deuse T, Reichenspurner H, Fischbein M P, Robbins R C, Pelletier M P. Stem cell transplantation: the lung barrier. Transplant Proc 2007; 39: 573-6.
Schmidt, et al. Closed head injury-an inflammatory disease? Brain Res. Rev. 2005; 48:388-399.
Sekiya I, Larson B L, Smith J R, Pochampally R, Cui J G, Prockop D J. Expansion of human adult stem cells from bone marrow stroma: conditions that maximize the yields of early progenitors and evaluate their quality. Stem Cells 2002; 20: 530-41.
Shlosberg D, Benifla M, Kaufer D, Friedman A. Blood-brain barrier breakdown as a therapeutic target in traumatic brain injury. Nat Rev Neurol 2010; 6: 393-403.
Suehiro E, Fujisawa H, Akimura T, Ishihara H, Kajiwara K, Kato 5, et al. Increased matrix metalloproteinase-9 in blood in association with activation of interleukin-6 after traumatic brain injury: influence of hypothermic therapy. J Neurotrauma 2004; 21:1706-11.
Szanto 5, Bardos T, Gal I, Glant T T, Mikecz K. Enhanced neutrophil extravasation and rapid progression of proteoglycan-induced arthritis in TSG-6-knockout mice. Arthritis Rheum 2004; 50: 3012-22.
Thau Zuckman, et al. The anti-inflammatory drug carprofen improves long-term outcome and induces gliogencesis after traumatic brain injury. J. Neurotrauma. 2012; 29:375-384.
Thurman D J, Alverson C, Dunn K A, Guerrero J, Sniezek J E. Traumatic brain injury in the United States: A public health perspective. J Head Trauma Rehabil 1999; 14: 602-15.
Uccelli A, Prockop D J. Why should mesenchymal stem cells (MSCs) cure autoimmune diseases? Curr Opin Immunol 2010; 22: 768-74.
Vlodaysky E, Palzur E, Soustiel J F. Hyperbaric oxygen therapy reduces neuroinflammation and expression of matrix metalloproteinase-9 in the rat model of traumatic brain injury. Neuropathol Appl Neurobiol 2006; 32: 40-50.
Wang X, Jung J, Asahi M, Chwang W, Russo L, Moskowitz M A, et al. Effects of matrix metalloproteinase-9 gene knock-out on morphological and motor outcomes after traumatic brain injury. J Neurosci 2000; 20: 7037-42.
Wisniewski H G, Hua J C, Poppers D M, Naime D, Vilcek J, Cronstein B N. TNF/IL-1-inducible protein TSG-6 potentiates plasmin inhibition by inter-alpha-inhibitor and exerts a strong anti-inflammatory effect in vivo. J Immunol 1996; 156:1609-15.
Wisniewski, H., Vilcek, J. Cytokine-induced gene expression at the crossroads of innate immunity, inflammation and fertility. Cytokine Growth Factor Reviews 2004; 15:129-146.
Wolfe M, Pochampally R, Swaney W, Reger R L. Isolation and culture of bone marrow-derived human multipotent stromal cells (hMSCs). Methods Mol Biol 2008; 449: 3-25.
Yong V W, Power C, Forsyth P, Edwards D R. Metalloproteinases in biology and pathology of the nervous system. Nat Rev Neurosci 2001; 2: 502-11.
Zhang, et al. Constitutive expression of inter-α-inhibitor (IαI) family proteins and tumor necrosis factor-stimulated gene-6 (TSG-6) by human amniotic membrane epithelial and stromal cells supporting formation of the heavy chain-hyaluronan (HC-HA) complex. J. Biol. Chem. 2012; 287:12433-12444.
Zhao J, Moore A N, Redell J B, Dash P K. Enhancing expression of Nrf2-driven genes protects the blood brain barrier after brain injury. J Neurosci 2007; 27:10240-8.
Zietlow R, Lane E L, Dunnett S B, Rosser A E. Human stem cells for CNS repair. Cell Tissue Res 2008; 331: 301-22.

What is claimed is:

1. A method of treating a brain injury or brain trauma in an animal comprising:
administering to said animal at least one inflammation modulatory or anti-inflammatory protein or polypeptide, or biologically active fragment, derivative, or analogue thereof, said at least one inflammation modulatory or anti-inflammatory protein or polypeptide, or biologically active fragment, derivative, or analogue thereof being administered in an amount effective to treat said brain injury or brain trauma in said animal.

2. The method of claim 1 wherein said at least one inflammation modulatory or anti-inflammatory protein or polypeptide or biologically active fragment, derivative, or analogue thereof, is tumor necrosis factor-α stimulating gene-6 (TSG-6) protein, or a biologically active fragment, analogue, or derivative thereof.

3. The method of claim 2 herein said TSG-6 protein or biologically active fragment, derivative, or analogue thereof is administered intravenously.

4. The method of claim 2 wherein said TSG-6 protein or biologically active fragment, derivative, or analogue thereof is administered directly to the site of said brain injury.

5. The method of claim 1 wherein said animal is a primate.

6. The method of claim 5 wherein said primate is a human.

7. The method of claim 1 wherein said at least one inflammation modulatory or anti-inflammatory protein or polypeptide, or biologically active fragment, derivative, or analogue thereof is administered to said animal in combination with at least one agent selected from the group consisting of antioxidants; free radical scavengers; ion channel blockers; NMDA antagonists; GABA agonists; neuroprotectants; anti-apoptotic agents; antagonists of cardiotonic steroids; proteinase inhibitors; inter-alpha-inhibitors; diuretics; and anti-seizure drugs.

8. The method of claim 7 wherein said at least one agent is an antagonist of cardiotonic steroids.

9. The method of claim 1 wherein said at least one inflammation modulatory or anti-inflammatory protein or polypeptide or biologically active fragment, derivative, or analogue thereof is administered in a total amount of from about 10 μg to about 100 μg.

10. The method of claim 9 wherein said at least one inflammation modulatory or anti-inflammatory protein or polypeptide or biologically active fragment, derivative, or analogue thereof is administered in a total amount of from about 50 μg to about 100 μg.

11. The method of claim 1 wherein said at least one inflammation modulatory or anti-inflammatory protein or polypeptide or biologically active fragment, derivative, or analogue thereof is administered within 24 hours of the infliction of said brain injury or brain trauma.

12. The method of claim 11 wherein said at least one inflammation modulatory or anti-inflammatory protein or polypeptide or biologically active fragment, derivative, or analogue thereof is administered within 6 hours of the infliction of said brain injury or brain trauma.

13. The method of claim 1 wherein said at least one inflammation modulatory or anti-inflammatory protein or polypeptide or biologically active fragment, derivative, or analogue thereof is administered at 6 hours after the infliction of said brain injury or brain trauma, and at 24 hours after the infliction of said brain injury or brain trauma.

14. The method of claim 13 wherein said inflammation modulatory or anti-inflammatory protein or polypeptide, or biologically active fragment, derivative, or analogue thereof is TSG-6 protein or a biologically active fragment, derivative, or analogue thereof, and said TSG-6 protein or biologically active fragment, derivative, or analogue thereof is administered in an amount of about 50 μg at 6 hours after the infliction of said brain injury or brain trauma, and is administered in an amount of about 50 μg at 24 hours after the infliction of said brain injury or brain trauma.
